(19) United States
(12) Patent Application Publication
(10) Pub. No.: US 2012/0109186 A1
(43) Pub. Date: May 3, 2012

(54) ARTICULATING LAPAROSCOPIC SURGICAL INSTRUMENTS

(76) Inventors: David A. Parrott, Cincinnati, OH (US); Benjamin T. Krupp, Wyoming, OH (US); Christoph L. Gillum, Middletown, OH (US); Christopher J. Matice, Bellbrook, OH (US); Louis P. Mingione, Chicago, IL (US)

(21) Appl. No.: 12/916,142

(22) Filed: Oct. 29, 2010

(51) Int. Cl. A61B 17/28

(52) U.S. Cl. 606/206

(57) ABSTRACT

An articulating laparoscopic instrument including a handle body, a shaft, an end effector, an operation mechanism, and an articulation mechanism. The end effector is connected to a shaft end. The operation mechanism includes a rod and an actuator. The rod is coupled to the end effector. Movement of the actuator relative to the handle body transfers a force onto the rod in a longitudinal direction to operate the end effector. The articulation mechanism includes a deflection assembly, first and second collar assemblies, first and second cables, a linkage, and an articulation actuator. The first and second collar assemblies are slidably disposed over the rod. The cables extend between the collar assemblies and the deflection assembly. Movement of the articulation actuator drives the first and second collar assemblies in opposite directions to cause a longitudinal deflection in the deflection assembly.

### BACKGROUND

The present disclosure relates to articulating laparoscopic surgical instruments. More particularly, it relates to articulating laparoscopic surgical instruments providing user-actuation and control over the operation and spatial positioning of an end effector carried by the instrument so as to be useful in performing, for example, single incision laparoscopic procedures.

There is a growing trend in laparoscopic surgery to be as minimally invasive as possible. This has pushed surgeons to perform procedures with fewer and smaller incisions. With more recent protocols, only one incision is made (in the umbilicus) through which all of the instrumentation and even the camera are inserted. While highly promising, this technique presents many obstacles including lack of triangulation, instrument reach, handle clashing, etc.

Various articulating laparoscopic surgical instruments have been developed in an attempt to address one or more of the above concerns. In general terms, an articulating laparoscopic surgical instrument includes an elongated shaft carrying an end effector (or “working end”) at the distal end. The end effector can assume various forms, such as scissors, graspers, needle holders, dissectors, clamps, etc. A portion of the shaft (typically proximate the end effector) can be caused to deflect or bend. A handle at the proximal end of the shaft affords user control over the end effector and the articulating shaft. When employed with laparoscopic procedures, articulating instruments allow the surgeon to regain triangulation during single port surgery by aiming the shaft of the surgical instrument slightly away and then curving the working end (or end effector) back toward the operative site. In addition, their longer lengths provide the surgeon the reach needed for organs further away from the umbilicus. Further, their low profile handles minimize handle clashing at the entrance site.

To be truly viable, the articulating laparoscopic instrument should afford user control, via actuators along the instrument's handle, over operation of the end effector, rotation of the end effector, articulation of the shaft, and rotation of the shaft. The mechanisms necessary to provide these multiple control features at the small scales associated with laparoscopic instrumentation are inherently intricate and dramatically increase the instrument's cost. While existing articulating laparoscopic surgical instruments may provide one or more of these features, they are limited to one-time use or are otherwise disposable because their design does not allow for proper cleaning and sterilization. Nor are they robust enough to stand up to repeated use. Due to the high cost, single-use nature of existing articulating laparoscopic instruments, a caregiver may unfortunately decide against purchasing or using such instruments. As a result, the single incision laparoscopic surgical procedures performed by the caregiver will be more complicated or even avoided.

In light of the above, a need exists for improved articulating laparoscopic instruments that facilitate desired surgeon control over instrument operation, articulation, and rotation.

### SUMMARY

Some aspects of the present disclosure relate to an articulating laparoscopic surgical instrument. The instrument includes a handle body, an elongated shaft, an end effector, an end effector operation mechanism, and an articulation mechanism. The elongated shaft extends from the handle body to a shaft end. The end effector is connected to the shaft end, and includes a first body movably associated with a second body. The end effector operation mechanism includes a rod and an end effector operation actuator. The rod defines a proximal portion maintained by the handle body and a distal portion extending from the handle body and through the shaft. A distal end of the rod is coupled to the end effector such that longitudinal movement of the rod causes the first body to move relative to the second body. In other words, the rod establishes a push/pull arrangement with respect to the end effector. The end effector operation actuator is movably coupled to the handle and is linked to the proximal portion of the rod. In this regard, movement of the end effector operation actuator relative to the handle body transfers a force onto the rod in a longitudinal direction. The articulation mechanism includes a deflection assembly, first and second collar assemblies, first and second cables, a linkage, and an articulation actuator. The deflection assembly is disposed over at least a segment of the distal portion of the rod, and is configured to bend and straighten the so-encompassed rod segment. The first and second collar assemblies are slidably disposed over the proximal portion of the rod, with the first collar assembly being longitudinally spaced from the second collar assembly. The first cable extends between the first collar assembly and the deflection assembly. Similarly, the second cable extends between the second collar assembly and the deflection assembly. The linkage interconnects first and second collar assemblies. The articulation actuator is coupled to the linkage and movably connected to the handle body. In this regard, the articulation mechanism is configured such that movement of the articulation actuator relative to the handle body moves the first and second collar assemblies in opposite directions to cause a longitudinal deflection in the deflection assembly via the cables. Thus, the articulating laparoscopic surgical instrument is highly useful in performing laparoscopic procedures, such as single incision laparoscopic procedures, providing a user with the ability to actuate the end effector and articulate the end effector relative to the handle body.

In some embodiments, the surgical instrument further includes a flush port fluidly coupled to a lumen of the shaft, thereby rendering the instrument reusable. In other embodiments, the collar assemblies each include first and second collar members, with the first collar member connected to the corresponding cable, and the second collar member connected to the linkage. In related embodiments, a shaft rotation knob is rotatably coupled to the handle body, and is rotatably fixed to the first collar members. With this construction, the shaft can be rotated relative to the handle body with rotation of the actuator knob, and the cables will follow the rotational movement via the first collar members. In yet other embodiments, the surgical instrument includes an end effector rotation mechanism including a thumb wheel connected to the rod in a manner permitting longitudinal movement of the rod. Rotation of the thumb wheel actuator effectuates rotation of the end effector relative to the shaft.

Other aspects in accordance with principles of the present disclosure relate to an articulating laparoscopic surgical instrument including a handle body, a shaft, an end effector, an end effector operation mechanism, and a knob operable to effectuate articulation and shaft rotation. The shaft extends from the handle body and is connected to the end effector at an opposite end. The end effector operation mechanism includes the rod and actuator as described above. A deflection assembly is disposed over at least a segment of the rod, and is configured to bend and straighten the segment. The knob is rotatably coupled to the handle body and rotationally fixed to the shaft. A pivot arm is disposed within a cavity formed by the knob, with the pivot arm being pivotably coupled to the shaft at a pivot point. With this construction, the pivot arm defines opposing, first and second end sections at opposite sides of the pivot point. A first cable extends between the first end section of the arm and the deflection assembly; similarly, a second cable extends between the second end section and the deflection assembly. The instrument is configured such that longitudinal movement of the knob relative to the shaft causes the pivot arm to pivot about the pivot point and apply opposing forces onto the cables, causing a longitudinal deflection in the deflection assembly. Further, rotation of the knob is transferred to the shaft to cause rotational movement of the shaft relative to the handle body.

Yet other aspects in accordance with principles of the present disclosure relate to an articulating surgical instrument including a handle body, a shaft, an end effector, and end effector operation mechanism, and an articulation mechanism. The shaft extends from the handle body and is connected to the end effector at an opposite end thereof. The end effector operation mechanism includes the rod and end effector operation actuator as described above. The articulation mechanism includes a deflection assembly, a paddle, and first and second cables. The deflection assembly is disposed over at least a segment of the rod, and is configured to bend and straighten the segment. The paddle is pivotably coupled to an exterior of the handle body at a pivot point. With this construction, the paddle defines opposing first and second end sections at opposite sides of the pivot point. The first cable extends between the first end section and the deflection assembly, along a first side of the shaft. Similarly, the second cable extends between the second end section and the deflection assembly along an opposite, second side of the shaft. With this construction, the articulation mechanism is configured such that pivoting of the paddle relative to the handle body applies opposing forces onto the cables, causing a longitudinal deflection of the deflection assembly.

### BRIEF DESCRIPTION OF THE DRAWINGS

FIG. 1 is a side view of an articulating laparoscopic surgical instrument in accordance with principles of the present disclosure;

FIG. 2 is an enlarged perspective view of a distal portion of the instrument of FIG. 1, illustrating a deflection assembly in an articulated state;

FIGS. 3A-3D are side views of a distal region of another embodiment surgical instrument in accordance with the present disclosure, illustrating articulation of a deflection assembly;

FIG. 4A is a top view of a distal region of another instrument, illustrating another embodiment deflection assembly in accordance with principles of the present disclosure;

FIG. 4B is a perspective view of another deflection assembly, and useful with instruments of the present disclosure;

FIG. 4C is a perspective view of another deflection assembly useful with instruments of the present disclosure;

FIG. 5 is a perspective view of a handle assembly useful with the instrument of FIG. 1, with portions shown in cross-section;

FIG. 6 is an enlarged, plan view of a distal region of the instrument of FIG. 1, illustrating coupling between an end effector and a rod component;

FIG. 7A is an enlarged rear perspective view of the handle assembly of FIG. 5;

FIG. 7B is an enlarged perspective view of a portion of the handle assembly of FIG. 5;

FIG. 8 is a side view of the handle assembly of FIG. 5 and illustrating operation of an articulation mechanism;

FIGS. 9A and 9B are side views of the instrument of FIG. 1 and illustrating rotation of an outer shaft component;

FIG. 10A is a simplified, perspective view of a portion of another embodiment articulating laparoscopic surgical instrument in accordance with principles of the present disclosure;

FIG. 10B is a simplified, schematic illustration of components of the instrument of FIG. 10A;

FIG. 11A is a perspective view of another handle assembly useful with the instrument of FIGS. 10A and 10B;

FIG. 11B is a perspective view of another handle assembly useful with the instrument of FIGS. 10A and 10B;

FIG. 11C is a simplified perspective view of another handle assembly useful with the instrument of FIGS. 10A and 10B;

FIG. 11D is a simplified perspective view of another handle assembly useful with the instrument of FIGS. 10A and 10B;

FIG. 12 is a perspective view of another articulating laparoscopic surgical instrument in accordance with principles of the present disclosure;

FIG. 13A is an enlarged side view of a handle assembly component of the instrument of FIG. 12, with portions removed;

FIG. 13B is a cross-sectional view of the handle assembly of FIG. 13A;

FIG. 13C is an enlarged view of a portion of the handle assembly of FIG. 13B;

FIG. 13D is an enlarged perspective view of a portion of the handle assembly of FIG. 13A, with portions shown in cross-section;

FIGS. 13E and 13F are side views of a portion of the handle assembly of FIG. 13B and illustrate optional use/cleaning modes;

FIG. 14A is a side view of a switch component of the handle assembly of FIG. 13A;

FIG. 14B is an end view of the switch component of FIG. 14A;

FIG. 15 is an exploded view of a portion of an end effector rotation mechanism useful with the handle assembly of FIG. 13A and including the switch of FIG. 14A;

FIG. 16 is a perspective view of another handle assembly useful with the instrument of FIG. 12;

FIG. 17A is an enlarged cross-sectional view of a portion of the handle assembly of FIG. 16;

FIG. 17B is a perspective view of the cross-section of FIG. 17A;

FIG. 17C is an enlarged perspective view of a portion of the handle assembly of FIG. 16;

FIG. 17D is an enlarged view of a portion of the handle assembly of FIG. 17A;

FIG. 18A is a perspective view of another articulating laparoscopic surgical instrument in accordance with principles of the present disclosure;

FIG. 18B is a simplified schematic illustration of components of the instrument of FIG. 18A;

FIG. 18C is an enlarged, perspective view of a handle assembly component useful with the instrument of FIG. 18A;

FIG. 19 is a perspective view of a portion of another articulating laparoscopic surgical instrument in accordance with principles of the present disclosure;

FIG. 20A is a side view of a portion of another articulating laparoscopic surgical instrument in accordance with principles of the present disclosure; and

FIG. 20B is a simplified schematic illustration of components of the instrument of FIG. 20A.

### DETAILED DESCRIPTION

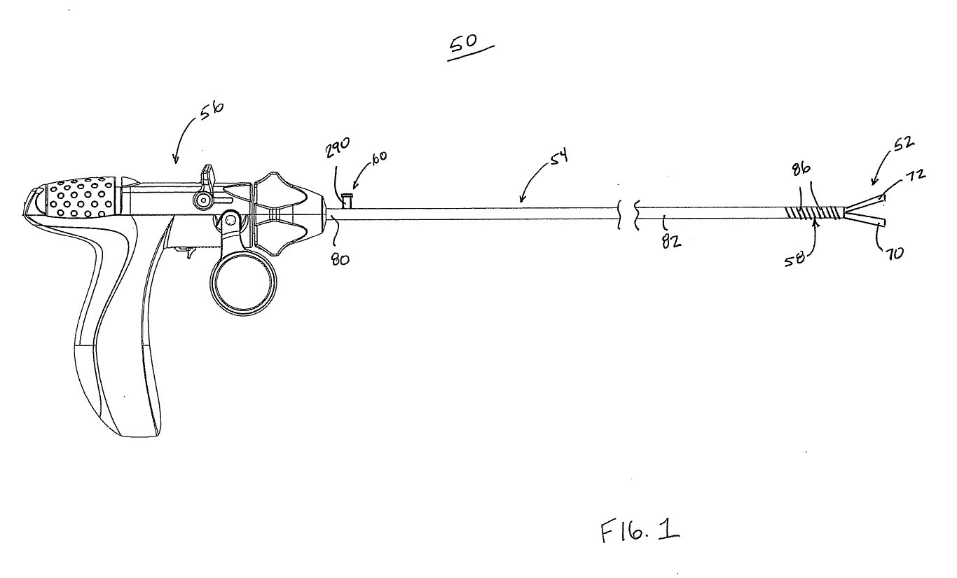

One embodiment of an articulating laparoscopic surgical instrument 50 in accordance with principles of the present disclosure is shown in FIG. 1. The surgical instrument 50 includes an end effector 52, a shaft 54, and a handle assembly 56. Details on the various components are provided below. In general terms, however, the shaft 54 extends from the handle assembly 56 and maintains or is connected to the end effector 52. A deflection assembly 58 is formed or carried by the shaft 54, and is configured to bend/articulate and straighten as described below. The handle assembly 56 is shaped for ergonomical grasping by a single hand of the user, and includes components of various mechanisms allowing a user to operate the end effector 52, rotate the end effector 52 relative to the shaft 54, articulate the deflection assembly 58, and optionally rotate the shaft 54. In some embodiments, the instrument 50 further includes or forms a flush port assembly 60 through which internal cleaning and sterilization of portions of the instrument 50 (e.g., a lumen of the shaft 54) can be performed. Thus, in some embodiments, not only does the instrument 50 provide a user with all operational control desired for single incision laparoscopic procedures (e.g., end effector operation and rotation, and shaft articulation and rotation), but also is reusable.

The end effector 52 can assume various forms useful with laparoscopic surgical procedures, such as scissors, grasper, clamp, dissector, needle holder, etc. In more general terms, the end effector 52 includes first and second bodies 70, 72, with at least the first body 70 being movably coupled relative to the second body 72. This movable coupling can be effectuated in various forms, such as by a pinned or pivoting interface, a cammed interface, various linkages, etc., as are known to those of skill in the art. Regardless, the end effector 52 is configured for connection with one or more additional components of the instrument 50 in a manner that facilitates operation of the end effector 52 (e.g., user-caused and controlled spatial arrangement of the first body 70 relative to the second body 72). In light of the wide variety of different end effector constructions implicated by the surgical instruments of the present disclosure, “operation” of the end effector is in reference to the movement(s) conventionally associated with the particular end effector design. Thus, “operation” of a scissors, grasper, or clamp-type end effector includes opening and closing of two opposing jaws relative to one another. Other types of end effectors entail differing movements, and the present disclosure is not limited to any particular end effector design or corresponding operative movements.

The shaft 54 can also assume various forms appropriate for delivery through a conventional trocar (e.g., the shaft 54 has a maximum diameter of less than 5 mm in some embodiments), and is generally tubular in shape. The tubular shaft 54 defines a proximal portion 80 and an intermediate portion 82. The proximal portion 80 is mounted to the handle assembly 56, with the intermediate portion 82 extending distal the handle assembly 56. In some constructions, the deflection assembly 58 is considered to be “part” of the shaft 54, and thus defines a distal portion of the shaft 54. Alternatively, the deflection assembly 58 can be entirely separate from the shaft 54 (e.g., is directly or indirectly assembled to and extends from the intermediate portion 82). Regardless, the intermediate portion 82 is relatively rigid, whereas the deflection assembly 58 is configured to reversibly articulate/bend and straighten in response to an applied force or tension as described below.

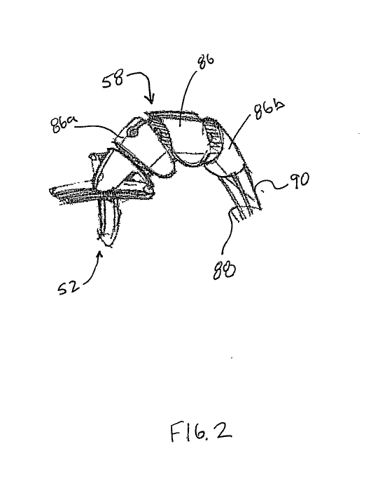

The deflection assembly 58 can have various formats now known or in the future developed, capable of providing selective bending or articulation (e.g., articulation up to 100 degrees). For example, the deflection assembly 58 can include a series of small, separated segments 86 (illustrated schematically in FIG. 1). The segments 86 can be exteriorly exposed or retained within an outer sheath. As shown in greater detail in FIG. 2, in some embodiments the segments 86 are not physically connected or pinned to one another, but instead are held in contact by opposing cables 88, 90. The cables 88, 90 are configured to apply necessary tension onto the deflection assembly 58, and can be metal wires, braids, flat bands, etc. The cables 88, 90 are attached to a distal-most segment 86a and pass through the remaining segments 86 in a manner permitting the remaining segments 86 to slide relative to the cables 88, 90. As a point of reference, a proximal-most segment 86b is immediately proximate (e.g., attached to) the shaft intermediate portion 82 (FIG. 1).

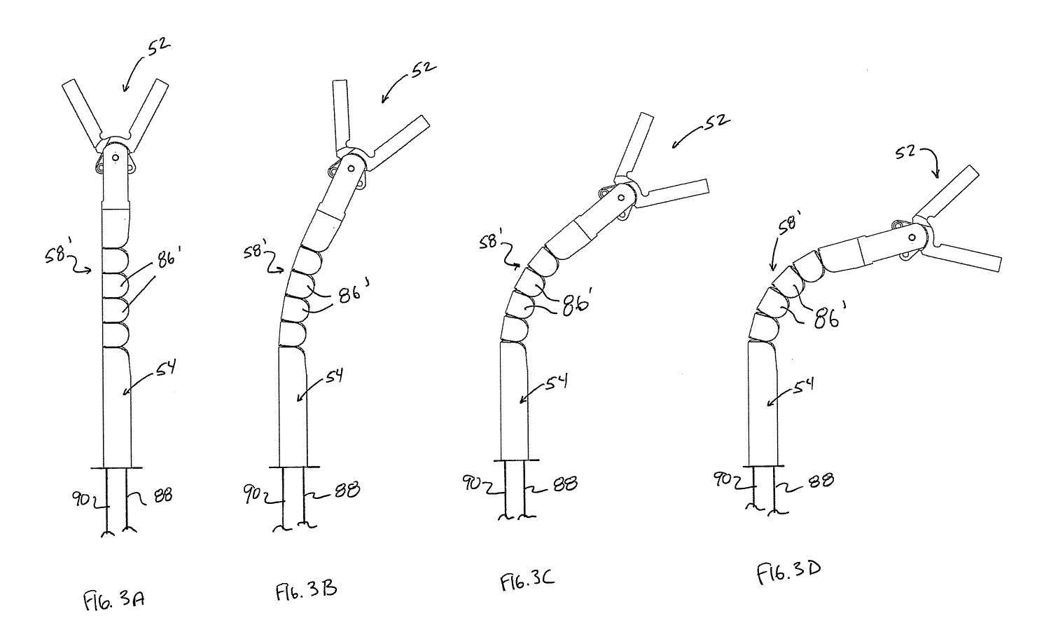

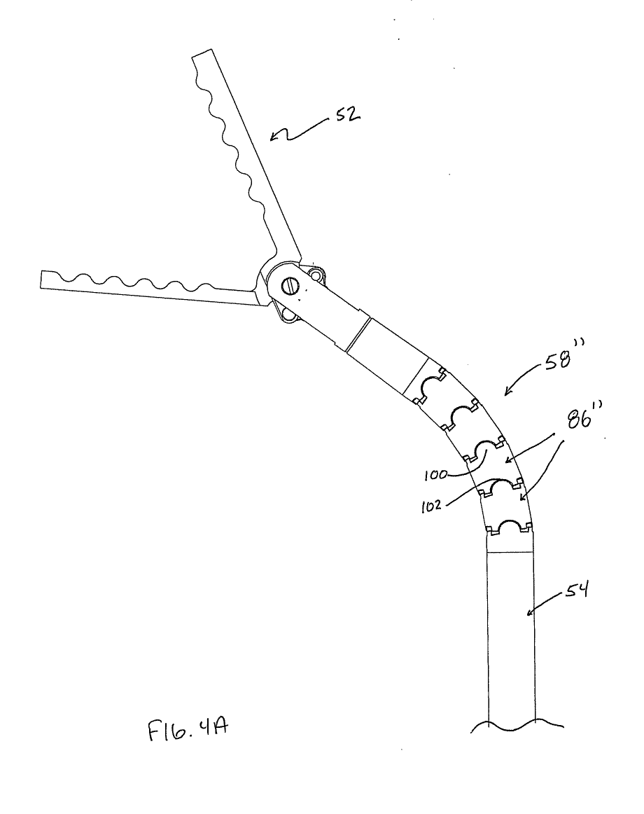

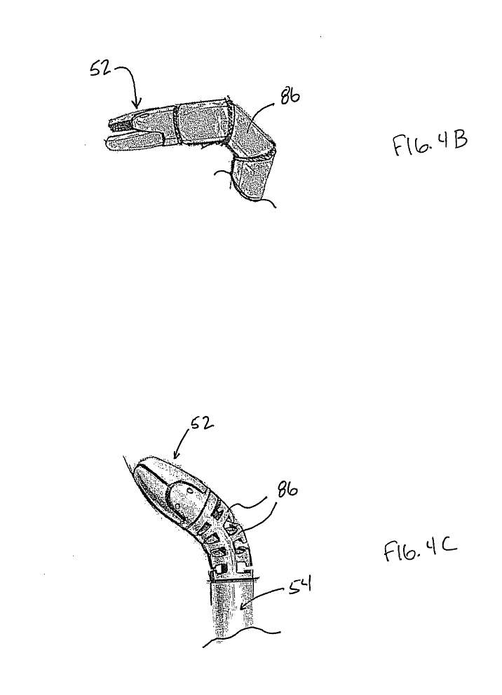

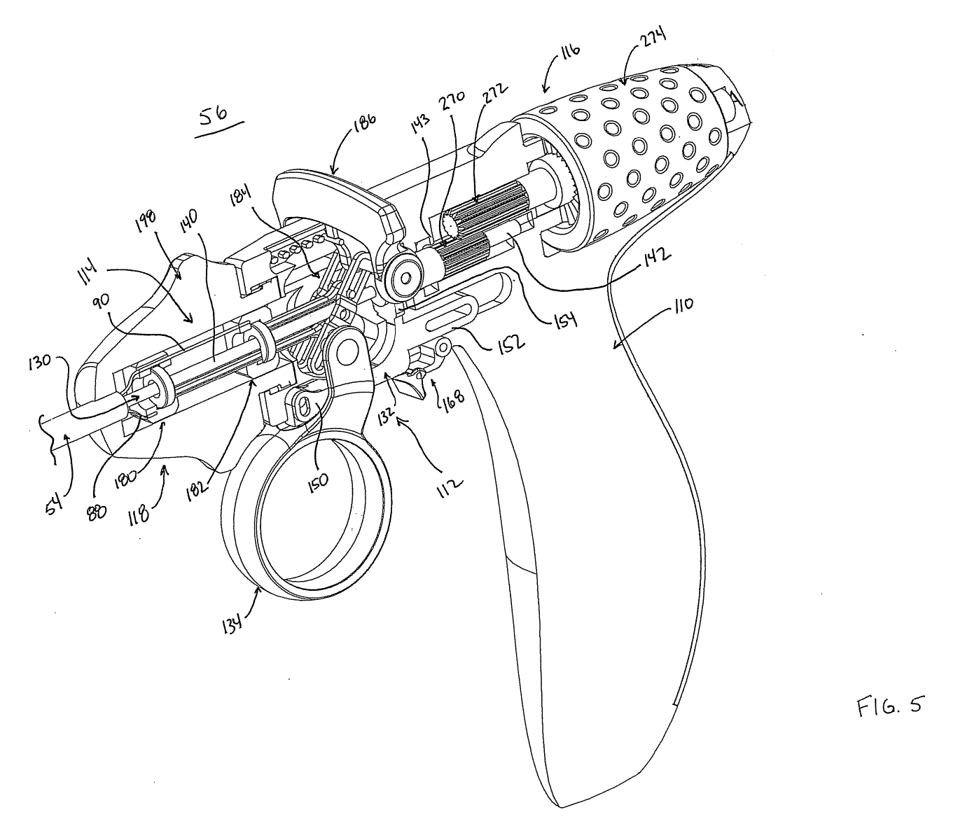

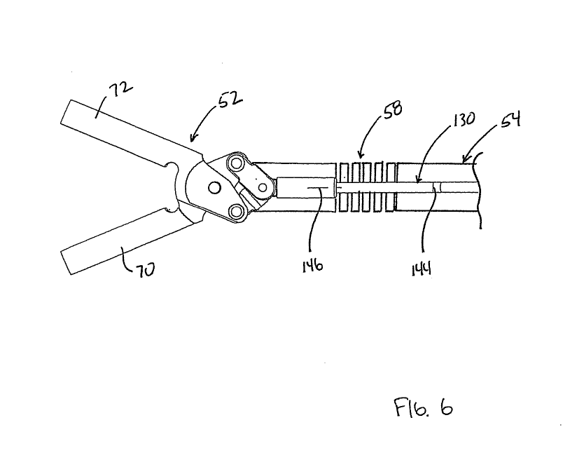

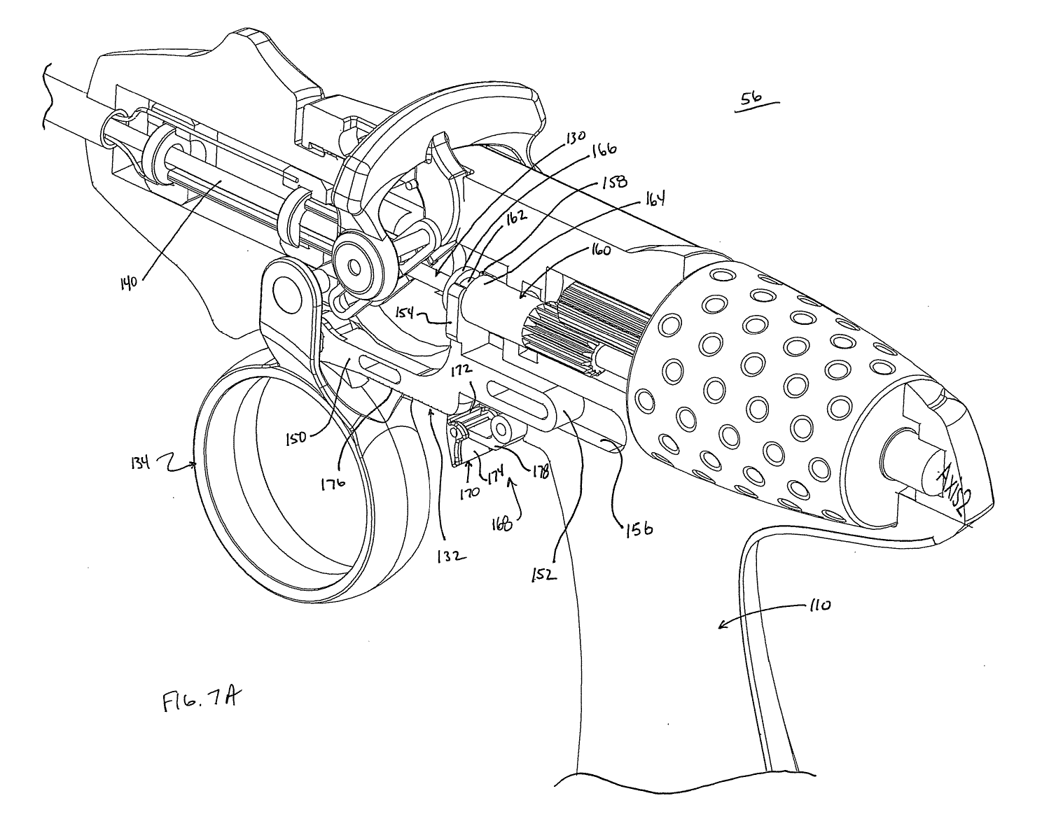

Articulation or bending of the deflection assembly 58 is achieved by pulling on the first (e.g., ventral) cable 88 (while possibly lessening tension in the second cable 90), whereas straightening of the deflection assembly 58 is achieved by pulling on the second (e.g., dorsal) cable 90 (while possibly lessening tension in the first cable 88). The so-applied tension or force is transferred to the distal-most segment 86a, causing the remaining segments 86 to collectively move or pivot relative to one another along the “side” at which the tension is applied. The segments 86 can assume various forms conducive to this articulation technique, with the cables 88, 90 providing movement-inducing tension regardless of whether the segments 86 are directly connected or pinned to one another. For example, the segments 86 can taper in shape toward the first cable 88. In a related alternative embodiment deflection assembly 58′ shown in FIGS. 3A-3D, the segments 86′ can have a generally U-shape, and roll relative to one another with tensioning or pulling of the cables 88, 90. In yet another alternative embodiment deflection assembly 58″ shown in FIG. 4A, the segments 86″ each form a male end 100 and a female end 102 that are correspondingly sized and shaped for articulating contact with one another (e.g., the male end 100 of a first segment 86″ rotatably nests within the female end 102 of an immediately adjacent segment 86″). In yet other constructions, the segments 86 are physically connected (e.g., angled cylinders of FIG. 4B) or can be integrally formed as a homogenous body as reflected in FIG. 4C.

Returning to FIG. 1, regardless of an exact construction, the deflection assembly 58 can be located immediately proximal the end effector 52 (sometimes referred to as a “wrist” of the instrument 50), or can be longitudinally spaced from the end effector 52. As a point of reference, the term “longitudinal” as used throughout the present disclosure is in reference to or based upon the linear central axis of the shaft 54.

The handle assembly 56 maintains various components useful for effectuating desired operation of the instrument 50. As implicated by the above explanations, the surgical instruments of the present disclosure are in no way limited to any particular end effector or shaft/deflection assembly design. Rather, some inventive features of the present disclosure relate to the handle assembly and corresponding operative mechanisms. While the handle assemblies/mechanisms can also assume various forms, some embodiments can be premised upon one or more common characteristics. The following descriptions of various handle assemblies in accordance with principles of the present disclosure are, in some respects, grouped by one or more common features.

Handle Assembly with Sliding Collars

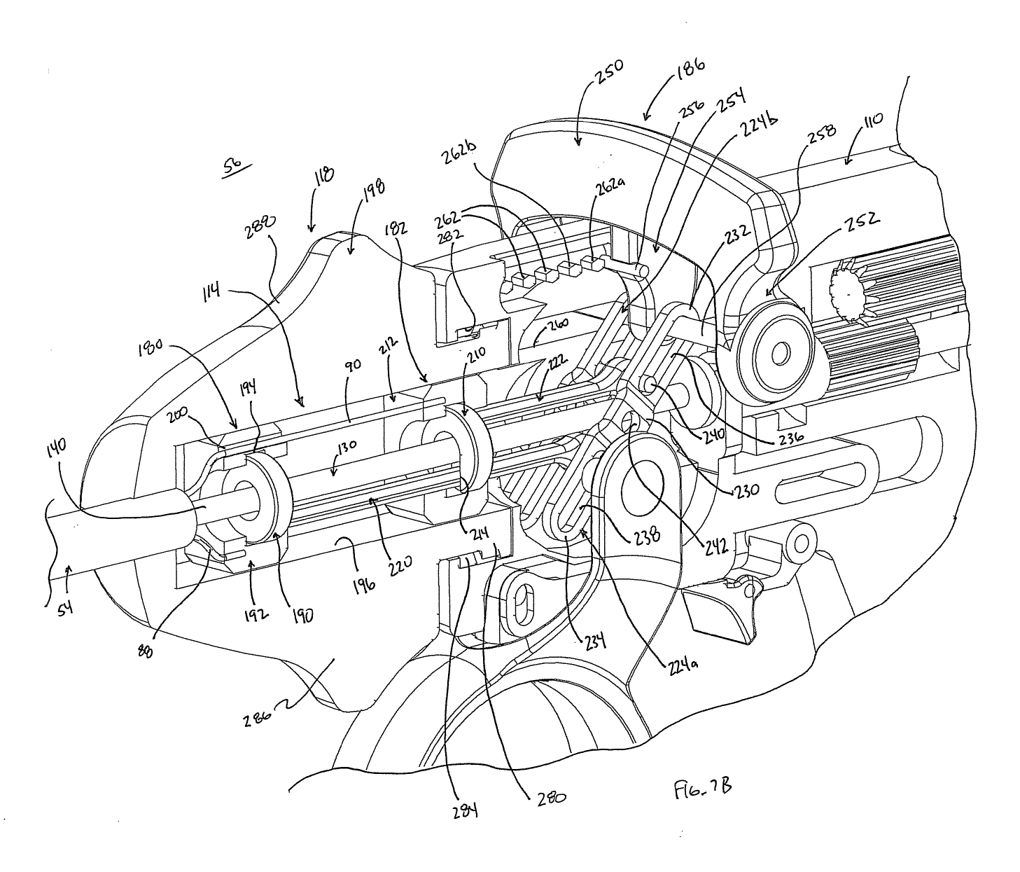

FIG. 5 illustrates one embodiment of the handle assembly 56 useful with the surgical instrument 50 in greater detail, including various internal components. In particular, the handle assembly 56 includes a handle body 110, an end effector operation mechanism 112 (referenced generally), an articulation mechanism 114 (referenced generally), an end effector rotation mechanism 116, and a shaft rotation mechanism 118. In general terms and with additional reference to FIG. 1, the end effector operation mechanism 112 facilitates operation of the end effector 52 by a user. The articulation mechanism 114 is operable to effectuate deflection or bending, as well as straightening, of the deflection assembly 58. The end effector rotation mechanism 116 is operable to rotate the end effector 52 relative to the shaft 54. Finally, the shaft rotation mechanism 118 is operable to spatially rotate the shaft 54 relative to the handle body 110.

The handle body 110 is generally sized and shaped for convenient handling by a user (e.g., a single-handed grasping), and thus can have other shapes and/or sizes differing from those implicated by the figures. Further, the handle body 110 can form or incorporate one or more internal or external features that maintain or interact with a component(s) of one or more of the mechanisms 112-118. In some constructions, the handle body 110 is formed of a surgically safe and sterilizable material, such as a molded plastic. The handle body 110 optionally includes two or more sections that are separately formed and subsequently assembled. In the view of FIG. 5, a half section of the handle body 110 is removed to better illustrate internal components of the handle assembly 56.

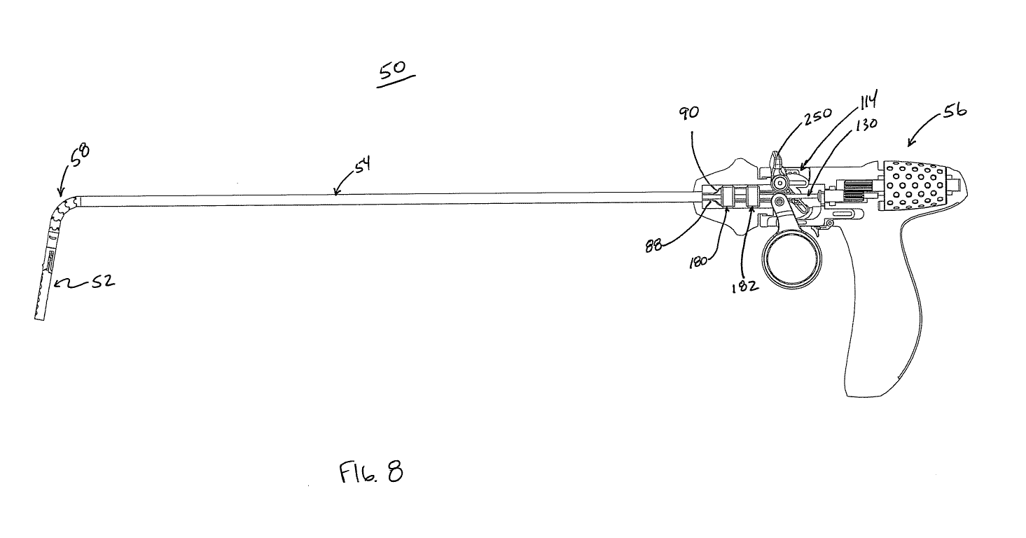

The end effector operation mechanism 112 includes a rod 130, a slide body 132, and an end effector operation actuator 134. The rod 130 forms or defines a proximal portion 140 terminating at a proximal end 142. The proximal portion 140 is slidably and rotationally maintained relative to the handle body 110 by one or more internal support surfaces 143. As shown, the rod 130 extends distally beyond the handle body 110, and is slidably received within the shaft 54. With additional reference to FIG. 6, a distal portion 144 of the rod 130 is disposed within the deflection assembly 58 (drawn schematically in FIG. 6 for ease of illustration), and terminates at a distal end 146 that is coupled to the end effector 52. At least the distal portion 144 exhibits sufficient flexibility to bend/straighten in response to corresponding forces applied by the deflection assembly 58. The distal portion 144 thus follows the shape or curvature defined by the deflection assembly 58, and will not permanently deform with repeated bending and straightening. Coupling between the distal end 146 and the end effector 52 can be achieved in various manners, and is more generally described as establishing a push/pull type link with the end effector 52. More particularly, because the end effector 52 is longitudinally fixed relative to the shaft 54 and because the rod 130 will slide relative to the shaft 54, longitudinal movement of the rod 130 relative to the end effector 52 causes the first body 70 to move relative to the second body 72. Thus, for example, the distal end 146 can be configured for coupling to a pivot point or cam structure provided with the end effector 52.

The rod 130 can be homogenously formed of a durable yet flexible material such as Nitinol™ or other material(s) that are robustly capable of repeated bending/straightening along the distal portion 144. In other constructions, the rod 130 can consist of two (or more) discrete materials, such as the proximal portion 140 formed of stainless steel and the distal portion 144 (or section of the distal portion 144 otherwise disposed within the deflection assembly 58) formed of Nitinol. The durable yet flexible construction of the rod 130 is sufficient to not only accommodate the articulation/bending described above, but also the axial compression/extension and torsion forces encountered during use of the instrument 50. For example, the rod 130 can be capable of maintaining its structural integrity in the presence of a tension force on the order of 150 lbf, a compression force on the order of 30 lbf, and a torsion force on the order of 0.41 in-lbf. Further, material(s) selected for the rod 130 are optionally able to maintain their structural integrity when subjected to repeated sterilization.

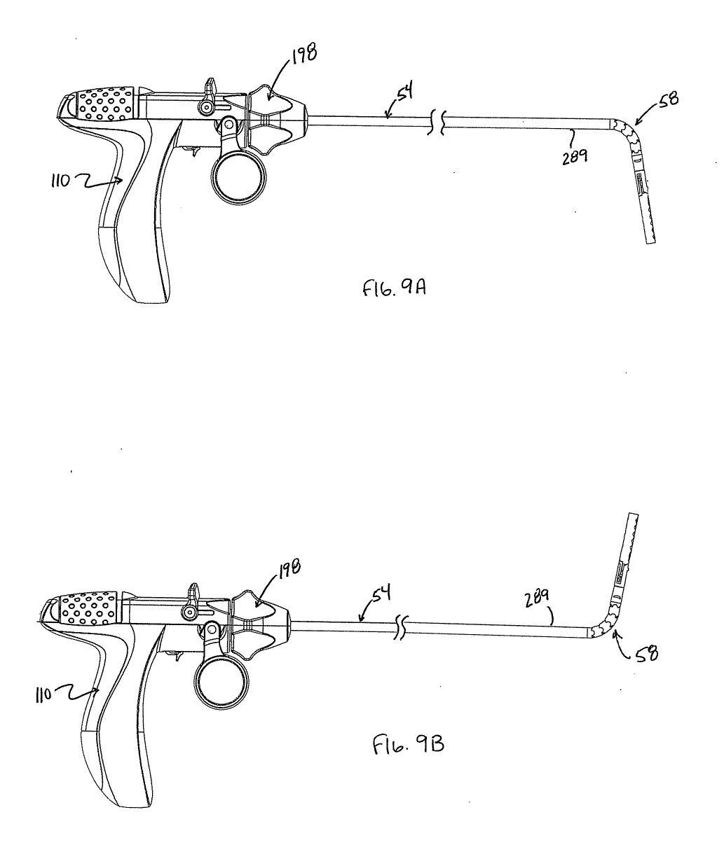

Returning to FIG. 5, longitudinal movement of the rod 130 relative to the shaft 54 and the handle body 110 (and thus operation of the end effector 52 (FIG. 6)) is effectuated via the slide body 132 and the end effector operation actuator 134. The slide body 132 forms or defines a leading leg 150, a trailing leg 152, and a flange 154. As best shown in FIG. 7A, the leading leg 150 is configured for coupling with the end effector operation actuator 134 as described below. The trailing leg 152 projects from the leading leg 150, and is sized to be slidably captured within a slot 156 defined by the handle body 110. Finally, the flange 154 projects upwardly relative to the legs 150, 152, and forms an open aperture 158 (referenced generally) sized to rotatably capture a corresponding segment of the rod 130. More particularly, a spindle body 160 is assembled to, or formed by, the rod 130 along the proximal portion 140 thereof, and defines a circumferential bearing surface 162 between opposing first and second hubs 164, 166. The bearing surface 162 is sized to be rotatably received within the aperture 158, with a diameter of the hubs 164, 166 being greater than a diameter of the aperture 158. A longitudinal spacing between the hubs 164, 166 is at least slightly greater than a thickness of the flange 154. With this construction, then, while the rod 130 can rotate relative to the flange 154 via the aperture 158/bearing surface 162 interface, longitudinal movement of the slide body 132 relative to the handle body 110 is transferred onto the rod 130 via an abutting interface between the flange 154 and the hubs 164, 166. For example, longitudinally rearward or proximal movement of the slide body 132 applies a corresponding force onto the first hub 164, causing the rod 130 to longitudinally move in the proximal direction. Conversely, a distal or forward movement of the slide body 132 is transferred by the flange 154 onto the second hub 166, causing a corresponding distal longitudinal movement of the rod 130.

Returning to FIG. 5, forward and rearward sliding of the slide body 132 relative to the handle body 110 is effectuated via a user-applied force the end effector operation actuator 134. In this regard, the actuator 134 can assume various forms, and in some embodiments has the trigger-like shape shown. The trigger actuator 134 is pivotably coupled to the handle body 110, generating an ergonomical pistol grip configuration. Other end effector operation actuator 134 constructions are also contemplated as described below, as well as differing mounting arrangements with the handle body 110. In general terms, user-caused, movement of the actuator 134 relative to the handle body 110 is transferred onto the slide body 132, and in turn onto the rod 130. As described above, forward or rearward movement of the rod 130 is transferred to the end effector 52 (FIG. 6), causing a change in the operational arrangement of the end effector 52.

The end effector operation mechanism 112 optionally further includes a locking device 168 configured to selectively lock the slide body 132 relative to the handle body 110 (and thus hold the rod 130 at a desired longitudinal position relative to the handle body 110, that in turn maintains a selected operative arrangement of the end effector 52 (FIG. 6)). As best shown in FIG. 7A, in one embodiment, the locking device 168 includes a locking body 170 forming a lip 172 and a tab 174. The lip 172 is configured to engage a toothed surface 176 (referenced generally) formed along the slide body 132. The locking body 170 is pivotably associated with the handle body 110 (e.g., by a clearance arm 178 extending from the handle body 110). A position of the slide body 132 (and thus a longitudinal position of the rod 130) relative to the handle body 110 can be temporarily locked by a user pressing the tab 174 to rotate the locking body 170, bringing the lip 172 into engagement with the toothed surface 176. Rotating the locking body 170 in the opposite direction releases the slide body 132.

Returning to FIG. 5, the articulation mechanism 114 is configured to accommodate the above-described longitudinal movement of the rod 130, as well as to effectuate desired bending or straightening of the deflection assembly 58 (FIG. 1). The articulation mechanism 114 includes, in some embodiments, a first collar assembly 180, a second collar assembly 182, a linkage 184 (referenced generally), and an articulation actuator 186. The cables 88, 90 are shown in FIG. 5, and can be considered as parts of the articulation mechanism 114. Similarly, in some constructions, the deflection assembly 58 described above is considered a component of the articulation mechanism 114. Regardless, and in general terms, the collar assemblies 180, 182 are connected to a corresponding one of the cables 88, 90, and are operable to simultaneously increase or decrease tension the cables 88, 90 in an opposing fashion in response to movement of the articulation actuator 186 via the linkage 184.

The first and second collar assemblies 180, 182 can be generally identical, and are slidably disposed about the proximal portion 140 of the rod 130. As identified in FIG. 7B, the first collar assembly 180 is maintained distal the second collar assembly 182, and includes first and second collar members 190, 192. The first collar member 190 is a ring-like body, and is coaxially disposed over the rod 130. An inner diameter of the first collar member 190 is slightly greater than a diameter of the rod 130 (at least along the region of interface between the rod 130 and the first collar member 190) so as to permit the rod 130 to freely rotate, and longitudinally slide, relative to the first collar member 190. The first collar member 190 is attached to a component of the linkage 184 as described below. The second collar member 192 forms a groove 194 within which the first collar member 190 is rotatably captured. For reasons made clear below, an exterior shape of the second collar member 192 corresponds with a shape of an internal bore 196 defined by a knob 198 provided with the handle assembly 56. With this construction, the second collar member 192 rotates relative to the first collar member 190 with rotation of the knob 198. However, the second collar member 192 can longitudinally slide within the internal bore 196. The first cable 88 is fixed to the second collar member 192 such that movement of the second collar member 192 is directly transferred to the first cable 88. In addition, the second collar member 192 forms a passage 200 sized to slidably receive the second cable 90. As shown, the second cable 90 extends through the passage 200, and is attached to the second collar assembly 182.

In many respects, the second collar assembly 182 is identical to the first collar assembly 180, and includes first and second collar members 210, 212. The first collar member 210 is disposed over the rod 130, and is configured to permit rotational and sliding movement of the rod 130 relative to the first collar member 210. Further, the first collar member 210 is attached to a component of the linkage 184, and forms a gap 214 (referenced generally) through which the linkage component otherwise attached to the first collar assembly 180 is slidably received. The second collar member 212 rotatably captures the first collar member 210, and is attached to the second cable 90. Finally, the second collar member 212 is slidably assembled within the bore 196 in a manner that provides sliding, rotational fixation between the knob 198 and the second collar member 192.

The linkage 184 includes a first drive arm 220, a second drive arm 222, and opposing pivot arms 224a, 224b. The arms 220-224b are structurally rigid, formed of a sterilizable material such as stainless steel. The first drive arm 220 is attached to, and extends from, the first collar member 190 of the first collar assembly 180; similarly, the second drive arm 222 is attached to, and extends from, the first collar member 210 of the second collar assembly 182. Each of the drive arms 220, 222 is pivotably coupled to the opposing pivot arms 224a, 224b. The pivot arms 224a, 224b are identical, with FIG. 7B illustrating the first pivot arm 224a as having or forming a central section 230 and opposing end sections 232, 234. The first end section 232 forms a slot 236; the second end section 234 also forms a slot 238. Though partially obstructed in the view of FIG. 7B, the second pivot arm 224b can have an identical construction. With this in mind, the second drive arm 222 is pivotably coupled to the pivot arms 224a, 224b via a pin 240 that is slidably captured within the slot 236 of the corresponding first end sections 232. Though hidden in the view of FIG. 7B, a separate pin similarly couples the first drive arm 220 with the slot 238 of the second end sections 234. Upon final assembly, the pivot arms 224a, 224b are rotatably maintained within the handle body 110, rotating about a common pivot point 242 established at the corresponding central section 230. For example, the handle body 110 can include or form posts (not shown) that rotatably maintain the central sections 230 at the pivot point 242. Regardless, the pivot point 242 is spatially arranged to pass through the rod 130 (and thus a central axis of the shaft 54), with the end sections 232, 234 extending at opposite sides of the rod 130 relative to the pivot point 242.

With the above construction, rotation of the pivot arms 224a, 224b imparts opposite direction forces onto the first and second drive arms 220, 222 via the pinned interface. For example, counterclockwise rotation of the pivot arms 224a, 224b (relative to the orientation of FIG. 7B) applies a distal or forward pushing force (in the longitudinal direction) onto the second drive arm 222, and an equal but opposite proximal or pulling force onto the first drive arm 220. The pins 240 can slide within the corresponding slot 236, 238 with rotation of the pivot arms 224a, 224b such that the generally parallel arrangement of the drive arms 220, 222 relative to the rod 130 is maintained. While two of the pivot arms 224a, 224b have been described, in other embodiments only a single pivot arm is provided.

A rotational orientation of the pivot arms 224a, 224b is dictated or controlled by the articulation actuator 186. The articulation actuator 186 is slidably maintained by the handle body 110 and is coupled to the linkage 184, and in particular to the pivot arms 224a, 224b. The articulation actuator 186 can assume various forms, and in some embodiments is akin to a switch having a thumb switch body 250 sized and shaped for interaction with a user's thumb or finger. Opposing legs 252 (one of which is visible in FIG. 7B) extend from the thumb switch body 250, as does an optional central guide wall 254. Upon final assembly, the legs 252 project along an exterior of the handle body 110. The guide wall 254 projects within an interior of the handle body 110, and is sized to be received within a spacing between the pivot arms 224a, 224b. The guide wall 254 can form a locking surface 256 described in greater detail below. A post 258 extends between the opposing legs 252 opposite the thumb switch body 250. The post 258 extends through channels 260 (one of which is shown in FIG. 7B) formed in the handle body 110, establishing a slidable coupling between the articulation actuator 186 and the handle body 110. Further, the post 258 is slidably captured within the slot 236 of the first end section 232 of each of the pivot arms 224a, 224b. Optionally, the post 258 passes through an aperture (hidden in the view of FIG. 7B) formed in the guide wall 254. Alternatively, a first post extends between a first one of the legs 252 and the guide wall 254, and a second post extends between the opposite leg 252 and the guide wall 254 in an identical fashion. Regardless, the post 258 translates a force applied to the thumb switch body 250 onto the pivot arms 224a, 224b (via the sliding interface within the slots 236). This force, in turn, is transferred to the collar assemblies 180, 182 via the drive arms 220, 222.

In some embodiments, the handle body 110 can form or include a series of protrusions 262 that selectively interface with the locking surface 256 in a manner that temporarily holds or retains the articulation actuator 186 at a selected position relative to the handle body 110, and thus holds or retains the pivot arms 224a, 224b at a selected rotational position. More particularly, in a normal state of the articulation mechanism 114, the articulation actuator 186 is positioned such that the locking surface 256 abuts one or two of the protrusions 262. For example, in the arrangement of FIG. 7B, the locking surface 256 abuts the first protrusion 262a, with this abutting interface preventing or temporarily “locking” the articulation actuator 186 at the position shown. The locking surface 256 will disengage or “ride over” the protrusion 262a in response to a sufficient pushing force applied to the thumb switch body 250, and subsequently lodges between the first and second protrusions 262a, 262b. In each of the locked positions implicated by FIG. 7B, then, the actuator 186 is temporarily locked relative to the handle body 110, effectively preventing movement of the collar assemblies 180, 182 unless a concerted effort is made by a user to move the switch actuator 186.

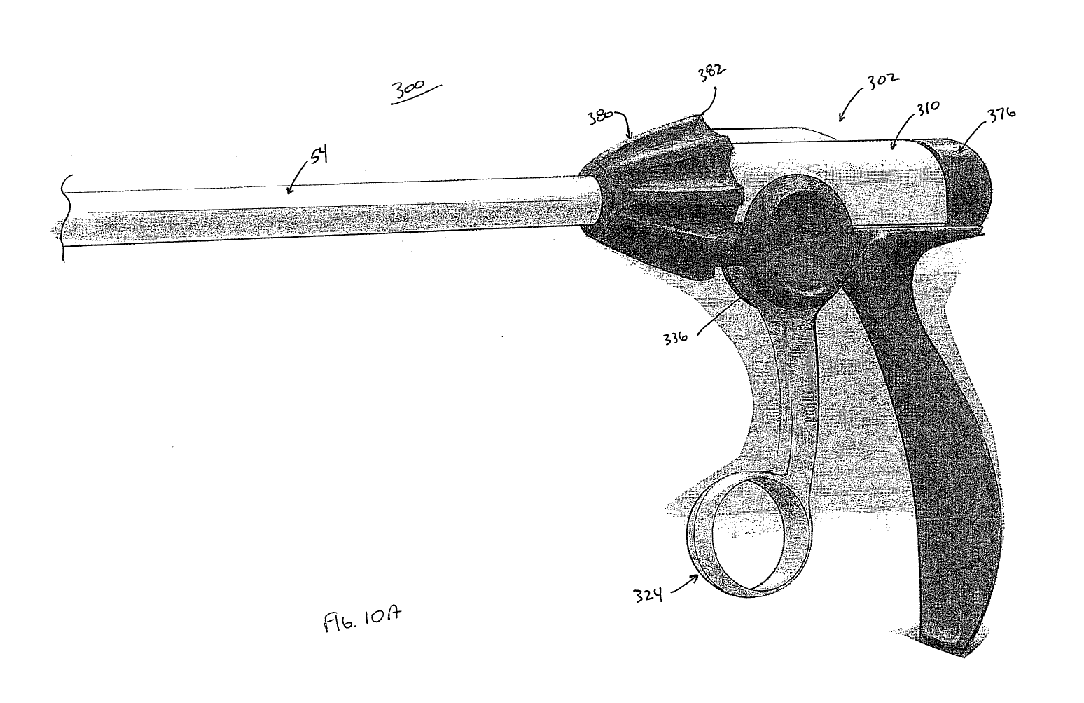

During use, and as indicated above, operation of the articulation mechanism 114 is initiated by a user-applied force at the thumb switch body 250. The so-applied force is translated onto the pivot arms 224a, 224b via the post 258. The pivot arms 224a, 224b rotate about the pivot point 242, simultaneously applying equal but opposite direction forces onto the drive arms 220, 222. The drive arms 220, 222, in turn, transfer the so-applied forces onto the corresponding collar assembly 180, 182. In particular, the first drive arm 220 transfers a force onto the first collar member 210 of the second collar assembly 182, with this force then being transferred to the second collar member 212 and thus the second cable 90 attached thereto. A similar interface is established between the second drive arm 222 and the first cable 88 via the collar members 190, 192 of the first collar assembly 180. By way of specific example, relative to the arrangement of FIG. 7B, a pushing force (distal direction) applied to the thumb switch body 250, causes the pivot arms 224a, 224b to rotate in a counterclockwise direction about the pivot point 242. This motion, in turn, effectuates a pulling (proximal) force onto the first cable 88 (i.e., increases a tension in the first cable 88) via the first collar assembly 180/first drive arm 220. Conversely, and essentially identical pushing (distal) force is applied to the second cable 90 (i.e., decreases a tension in the second cable 90) via the second collar assembly 182/second drive arm 222. As a result, the first and second collar assemblies 180, 182 are forced to move toward one another at a simultaneous rate and essentially identical distance as shown, for example, in FIG. 8. As a result, a pulling tension is applied to the first cable 88, and a corresponding release of tension is effectuated at the second cable 90. These altered tensions are transferred to the deflection assembly 58, resulting in a deflection or bend therein. The deflection assembly 58 can subsequently be straightened by a user-applied proximal force upon the thumb switch body 250. The collar assemblies 180, 182 are simultaneously directed away from one another, with the corresponding tension applied to the second cable 90 (and release of tension in the first cable 88) causing the deflection assembly 58 to revert back toward a more straightened arrangement. Notably, during operation of the articulation mechanism 114, the collar assemblies 180, 182 freely slide over the rod 130; thus, the deflection assembly 58 can be articulated and straightened without applying a pushing or pulling force directly upon the rod 130 (in a manner that might otherwise alter an operational arrangement of the end effector 52 as described above).

Returning to FIG. 5, the end effector rotation mechanism 116 includes, in some embodiments, the rod 130, first and second gears 270, 272, and an end effector rotation actuator 274. The first gear 270 is assembled to or formed by the proximal portion 140 of the rod 130. The second gear 272 is attached to and extends from the end effector rotation actuator 274, with teeth of the gears 270, 272 being configured for meshed engagement. The end effector rotation actuator 274 can assume various forms, and in some constructions is akin to a thumb wheel rotatably maintained by the handle body 110. As shown, upon final assembly, the end effector rotation actuator 274 is exteriorly exposed relative to the handle body 110 for convenient interface by a thumb or finger of the user's hand (otherwise grasping the handle body 110), with the second gear 272 meshingly engaged with the first gear 270. Rotation of the end effector rotation actuator 274 is transferred onto the rod 130 via the gears 270, 272. Rotation of the rod 130, in turn, is transferred to the end effector 52 (FIG. 1), causing the end effector 52 to rotate relative to the shaft 54.

Notably, the end effector rotation mechanism 116 is configured to permit desired operation of the end effector operation mechanism 112. In particular, and as described above, use of the end effector operation mechanism 112 generally entails longitudinal movement of the rod 130. With this in mind, a longitudinal length of the gears 270, 272 is sufficient so as to not only permit longitudinal movement of the rod 130 (i.e., teeth of the first gear 270 slide relative to teeth of the second gear 272), but also maintain meshed engagement between the gears 270, 272 throughout the entire possible range of longitudinal movement of the rod 130 relative to the second gear 272 (e.g., on the order of 0.15 inch). As a point of reference, FIG. 5 illustrates the rod 130 in a longitudinally forward-most position; with rearward longitudinal movement of the rod 130 in response to a squeezing force applied to the end effector operation actuator 134, the rod 130, and thus the first gear 270, will slide proximally or rearwardly along the second gear 272, and at all times meshed engagement is maintained between the gears 270, 272.

The optional shaft rotation mechanism 118 includes the knob 198 referenced above. The knob 198 serves as an actuator of the shaft rotation mechanism 118 and is affixed to the shaft 54. Further, the knob 198 is rotatably assembled to the handle body 110. For example, the knob 198 can form or define a base 280 configured to be rotatably captured within an annular receptacle 282 formed by the handle body 110. As shown, an optional gasket 284 (e.g., an O-ring) secures the base 280 within the receptacle 282, and serves to prevent longitudinal movement of the knob 198 relative to the handle body 110. A head 286 is further formed by the knob 198, and provides a contoured outer surface 288 sized and shaped for user interaction. With this construction, rotation of the knob 198 causes the shaft 54 to rotate relative to the handle body 110.

To minimize the possibility that the cables 88, 90 will twist during rotation of the shaft 54/knob 198, the knob 198 forms the internal bore 196, sized and shaped in accordance with the second collar members 192, 212 as described above with reference to FIG. 7B. More particularly, the bore 196 is configured to slidably capture the second collar members 192, 212 in a manner permitting longitudinal sliding of the second collar members 192, 212 along the bore 196, but preventing rotational movement between the knob 198 and the second collar members 192, 212. For example, in some constructions, the bore 196 and the second collar members 192, 212 have corresponding square or rectangular perimeter shapes. Other shapes are also acceptable. Regardless, the second collar members 192, 212 can longitudinally slide relative to the knob 198, but will rotate with rotation of the knob 198.

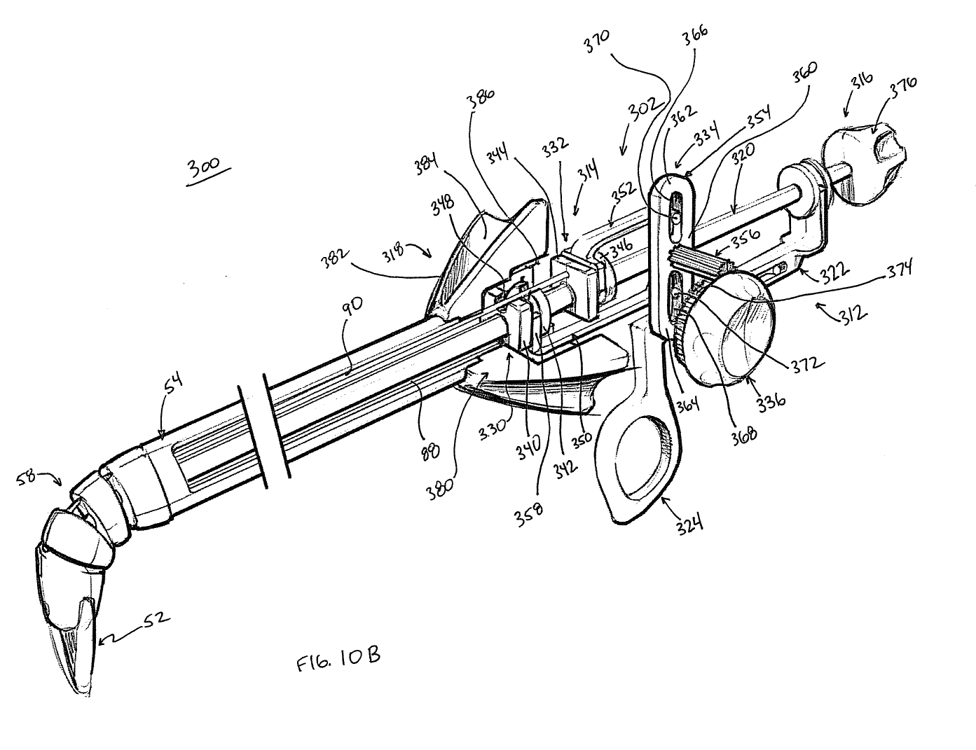

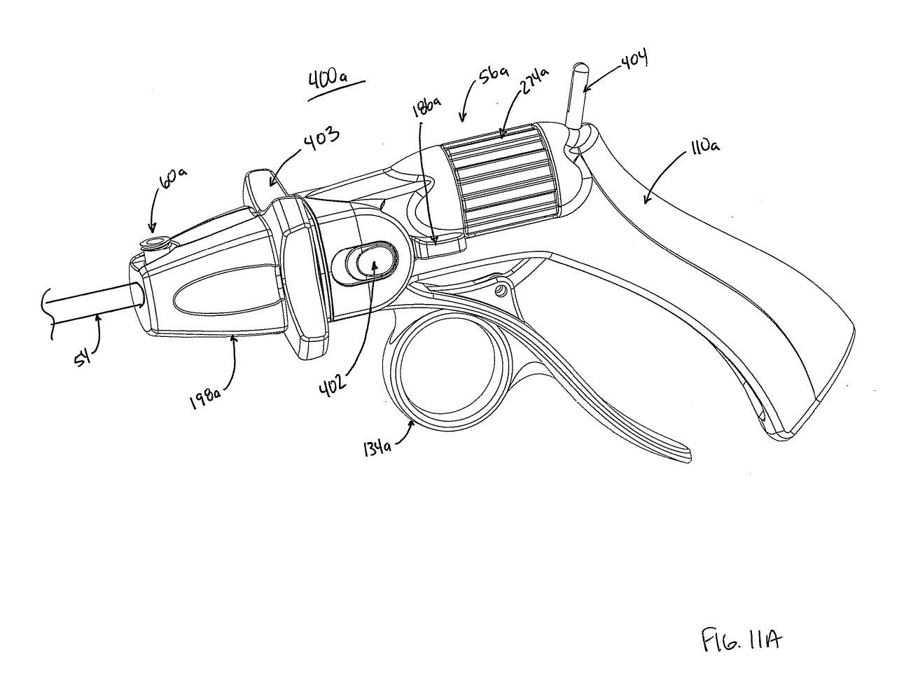

With the above construction, operation of the shaft rotation mechanism 118 includes the user applying a rotational force onto the knob 198. This rotational force is transferred to the shaft 54, resulting in rotation of the shaft 54. For example, with reference to FIG. 9A, in a first rotational position of the shaft 54 relative to the handle body 110, a bend in the deflection assembly 58 is spatially oriented in the direction shown. The inward curvature or bend is defined along an articulating side 289 of the shaft 54 (otherwise corresponding with the “side” of the shaft 54 along which the first cable 88 (FIG. 5) extends). With rotation of the knob 198, the shaft 54 rotates and the spatial orientation of the deflection assembly 58 is altered to the arrangement of FIG. 9B. The radius of curvature along the bend in the deflection assembly 58 is unchanged; however, the spatial location of the articulating side 289 has rotated 180°. Thus, even though bending of the deflection assembly 58 is effectuated at or along only one “side” of the shaft 54 (i.e., the articulating side 289), this side can be positioned at any rotational position (relative to the handle body 110) by rotating the shaft 54. As such, a user can rotate the articulating side 289 as desired without needing to physically rotate the handle body 110.

Notably, and returning to FIG. 7B, the second collar members 192, 212 rotate with rotation of the knob 198. Because the cables 88, 90 are affixed to corresponding ones of the second collar members 192, 212, the cables 88, 90 thus simultaneously rotate with the knob 198/shaft 54. Thus, the cables 88, 90 will not “bind” or twist with rotation of the shaft 54. Conversely, because the first collar members 190, 210 are rotationally separated from the corresponding second collar member 192, 212, the second collar members 192, 212 remain stationary during rotation of the knob 198. Thus, the longitudinal positions of the collar assemblies 180, 182 (and corresponding tensions applied to the cables 88, 90) do not change, meaning that the bend in the deflection assembly 58 as dictated by the collar assemblies 180, 182 is unaffected by rotation of the shaft 54. Finally, rotation of the knob 198/second collar members 192, 212 is mechanically isolated from the rod 130 such that the rod 130 (and thus the end effector 52) need not rotate with rotation of the knob 198. In the absence of any frictional resistance, then, the end effector 52 would effectively experience rotation relative to the shaft 54. However, various frictional forces at the coupling between the end effector 52 and the rod 130 and/or between the rod 130 and the deflection assembly 58 (when in an articulated position) may be high enough to cause the rod 130, and thus the end effector rotation actuator 274, to rotate along with the shaft 54. Handle assemblies of the present disclosure can be designed to ensure that the end effector 52 rotates with rotation of the shaft 54, or that the end effector 52 does not rotate with rotation of the shaft 54. In yet other embodiments, the handle assembly 56 incorporates additional features that permit a user to select whether or not the end effector 52 will rotate with rotation of the shaft 54.

During use and with reference to FIGS. 1 and 5, the articulating laparoscopic instrument 50, as well as any of the instruments described below, can be employed to perform a variety of laparoscopic procedures, including single incision laparoscopic procedures. Desired bending or articulation of the deflection assembly 58 is achieved by user manipulation of the articulation actuator 186. Operative movement of the end effector 52 (e.g., opening or closing the first body 70/second body 72) is achieved by user manipulation of the end effector operation actuator 134. The end effector 52 can be rotated by user manipulation of the end effector rotation actuator 274, whereas the shaft 64 can be rotated by user manipulation of the knob 198. Each of these actions can thus be performed with a single hand of the user otherwise grasping the handle assembly 56/handle body 110.

The optional flush port assembly 60 of FIG. 1 promotes cleaning and sterilization of portions of the instrument. For example, the flush port assembly 60 can include a flush port 290 attached to the shaft 54 proximate the handle assembly 56. The flush port 290 is fluidly open to a lumen of the shaft 54; cleaning liquid can thus be delivered through the flush port 290. In other embodiments, the flush port 290 can be assembled to other components of the instrument 50 (e.g., the knob 198 (FIG. 5)) otherwise fluidly open to the lumen of the shaft 54. Other features can be incorporated into the overall construction of the instrument 50 that facilitate cleaning and sterilization sufficient for re-use. For example, lumens defined by any component or sub-component of the instrument 50 can have a diameter of not less than 0.050 inch, optionally not less than 0.025 inch, to allow for cleaning No “hidden” internal crevices are generated by the instrument construction that might otherwise impede thorough removal of bioburden and/or accessing by conventional surgical instrument cleaning tools. The materials selected for all of the instrument components can withstand more than 300 sterilization/cleaning cycles. These optional features combine to render the instrument 50 to be re-usable. In other constructions, however, the flush port assembly 60 is omitted.

Cleaning and sterilization of the instrument 50 can be further enhanced by permitting selective separation of the deflection assembly segments 86. For example, the handle assembly 56 can be configured to provide a user-selected cleaning mode in which the tension in one or both of the cables 88, 90 (FIG. 5) is reduced or removed, generating slack in the cables 88, 90. This slack, in turn, allows adjacent ones of the articulating segments 86 to be more completely separated from each other (especially with constructions in which the articulating segments 86 are not directly pinned to one another) for cleaning. For example, with the handle assembly 56 of FIG. 7B, the handle assembly component(s) otherwise securing the pivot point 242 of the pivot arms 224a, 224b to the handle body 110 can be configured to establish a releaseable mounting. In a cleaning mode, the pivot arms 224a, 224b are released from direct mounting to the handle body 110 (i.e., the pivot arms 224a, 224b are not constrained to only pivoting movement relative to the handle body 110, and are allowed to move longitudinally). Forward or distal longitudinal movement of the pivot arms 224a, 224b (and thus of the linkage 184) slides the collar assemblies 180, 182 forward, thereby creating slack in the cables 88, 90. Once cleaning is complete, the handle assembly 56 is returned to the use mode reflected in the figures.

The optional cleaning mode features can be achieved with a variety of other constructions, and can be incorporated into any of the articulating laparoscopic surgical instruments/handle assemblies of the present disclosure. For example, a cam can be operatively associated with one or both of the cables 88, 90 (or a separate cam is provided for each of the cables 88, 90) between the deflection assembly 58 and an opposite, fixed end of the corresponding cable 88, 90. In a use mode, the cam is rotated into engagement with the cable(s) 88, 90 generating a “normal” level of tension in the cables 88, 90 (with the tension level subsequently being altered by operation of the articulation mechanism 114). In a cleaning mode, the cam is rotated out of engagement with the corresponding cable(s) 88, 90, thereby removing the cam-induced tension and creating slack in the cables 88, 90. In yet other embodiments, the handle assembly 56 is configured to require two (or more) concerted user input actions to “activate” the cleaning mode. Alternatively, or in addition, the handle assembly 56 is configured to receive a separate cleaning/sterilization “key,” and loosening of the cables 88, 90 occurs only upon insertion of the key. In yet other constructions, the cleaning mode actuator is located internal the handle body 110, and a separate access panel or door must be opened by a user before the cleaning mode can be effectuated.

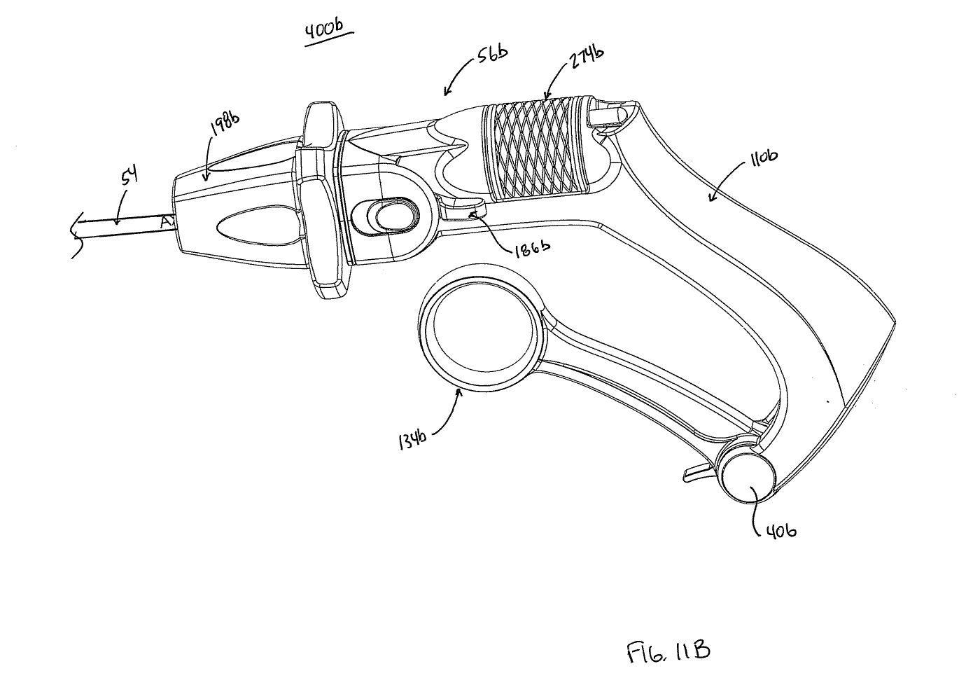

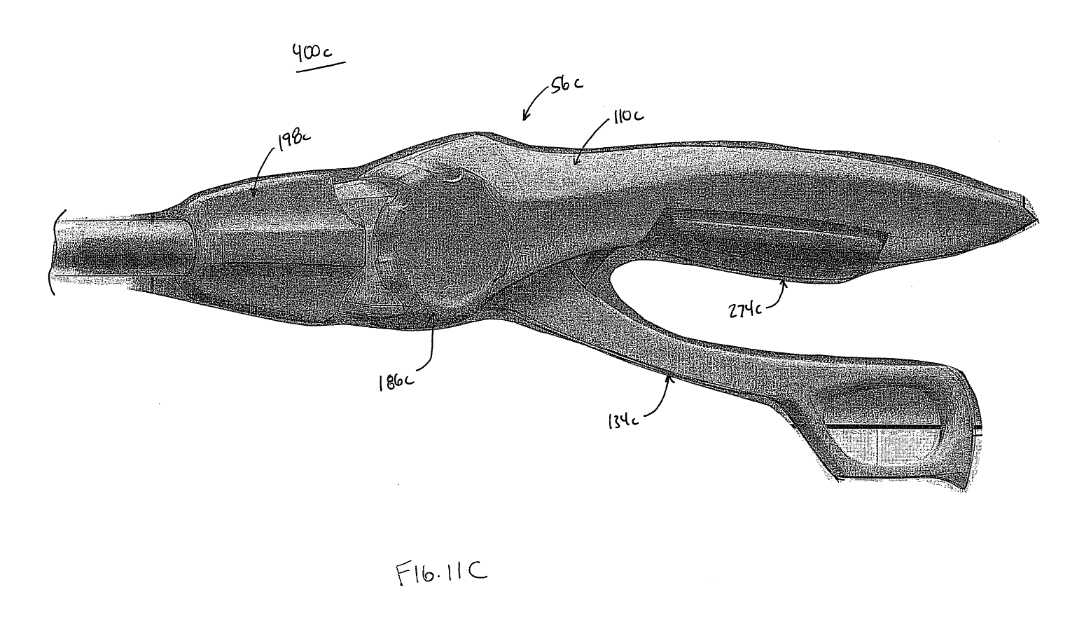

The handle assembly 56, and in particular the handle body 110 and the mechanisms 112-118 described above, can assume one or more other forms in accordance with principles of the present disclosure. For example, the sliding collar construction described above with respect to the articulation mechanism 114 can be implemented with other designs. FIG. 10A is a simplified illustration of another embodiment articulating laparoscopic surgical instrument 300 in accordance with principles of the present disclosure utilizing the sliding collar-based articulation mechanism. FIG. 10B illustrates internal components of the instrument 300 in a more schematic form. In general terms, the surgical instrument 300 is highly akin to the instrument 50 (FIGS. 1 and 5) described above, and includes the end effector 52, the shaft 54, and a handle assembly 302. The handle assembly 302 includes a handle body 310, an end effector operation mechanism 312, and articulation mechanism 314, an end effector rotation mechanism 316, and a shaft rotation mechanism 318.

The handle body 310 can assume any of the forms implicated by the present disclosure. In general terms, the handle body 310 is sized and shaped for convenient handling by a user (e.g., single-handed grasping) and maintains various components of the mechanisms 312-318 as described below.

The end effector operation mechanism 312 is akin to the end effector operation mechanism 112 (FIG. 5) described above, and includes a rod 320, a slide body 322, and an end effector operation actuator 324. The rod 320 can assume any of the forms described for the rod 130 (FIG. 5), and is coupled to the end effector 52 such that longitudinal movement of the rod 320 effectuates operation of the end effector 52. The slide body 322 is mounted to the rod 320 in a manner permitting rotation of the rod 320 relative to the slide body 322, but transferring a longitudinal force from the slide body 322 onto the rod 320. The end effector actuator 324 extends from the slide body 322, and can assume various forms appropriate for convenient user interface (e.g., the trigger-like shape shown). With this construction, a force applied to the end effector operation actuator 324 is transferred to the rod 320 via the slide body 322 to cause longitudinal movement of the rod 320 and thus operation of the end effector 52.

The articulation mechanism 114 includes first and second collar assemblies 330, 332, a linkage 334, and an articulation actuator 336. The first and second cables 88, 90 are attached to the first and second collar assemblies 330, 332, respectively, extend through the shaft 54, and are attached to the deflection assembly 58. Commensurate with previous explanations, in some constructions the cables 88, 90 and the deflection assembly 58 can be considered as components of the articulation mechanism 314.

The first and second collar assemblies 330, 332 are slidably disposed over the rod 320, and are separately connected to the linkage 334 as described below. In this regard, the collar assemblies 330, 332 can be substantially identical, with the first collar assembly 330 including opposing, spaced apart collar members 340, 342 connected to one another by a bearing ring (hidden in the view of FIG. 10B), and the second collar assembly 332 including opposing collar members 344, 346 connected by a bearing ring (not visible). The first cable 88 is attached to the first collar member 340 of the first collar assembly 330; the second cable 90 is attached to the first collar member 344 of the second collar assembly 332. As shown, the collar members 340, 342 of the first collar assembly 330 each form a notch 348 (identified for the first collar member 340) through which the second cable 90 extends. As described below, the collar assemblies 330, 332 can optionally incorporate other features that promote a desired interface with other components of the handle assembly 302.

The linkage 334 includes first and second drive arms 350, 352, a pivot arm 354, and a gear 356. The first drive arm 350 is coupled to the first collar assembly 330, for example via a fork 358 rotatably captured against the bearing ring (hidden) and between the corresponding collar members 340, 342. As a point of reference, a longitudinal spacing between the collar members 340, 342 is less than a thickness of the fork 358. With this construction, a longitudinal force placed upon the first drive arm 350 is transferred to the first collar assembly 330 via the fork 358 and effectuates longitudinal movement of the first collar assembly 330 relative to the rod 320. However, the first collar assembly 330 can rotate relative to the fork 358 at the bearing ring interface. The second drive arm 352 has a similar construction, and is longitudinally coupled to the second collar assembly 332 between the collar members 344, 346 in a manner permitting rotation of the second collar assembly 332 relative to the second drive arm 352.

Each of the drive arms 350, 352 are slidably coupled to opposite sides of the pivot arm 354. The pivot arm 354 includes or defines a central portion 360 and an opposing end portion 362, 364 each forming a slot 366, 368. A pin 370 formed by or extending from the first drive arm 350 is slidably captured within the slot 366 of the first end portion 362; similarly, a pin 372 provided by or with the second drive arm 352 is slidably captured within the slot 368 of the second end portion 364. The pivot arm 354 is rotatably maintained within the handle body 310, establishing a rotational pivot point at the central portion 360. An axis of the pivot point intersects the rod 320. Thus, the end portions 362, 364 extend from opposite sides of the rod 320. Finally, the gear 356 is rigidly attached to and extends from the central portion 360.

The articulation actuator 336 is rotatably mounted to the handle body 310, and is akin to a thumb wheel. In this regard, the articulation actuator 336 forms a toothed surface 374 that is arranged, upon final assembly, to mesh with the gear 356.

With the above construction, articulation (e.g., bending or straightening) of the deflection assembly 58 is effectuated by a user rotating the articulation actuator 336. This rotation is transferred to the pivot arm 354 via the gear 356. The pivot arm 354, in turn, is caused to rotate about the pivot point established at the central portion 300, imparting opposite direction forces onto the drive arms 350, 352. The drive arms 350, 352 are thus caused to move longitudinally in opposite directions (with the drive arm pin 370, 372 sliding within the corresponding pivot arm slot 366, 368), with this same movement being transferred to the corresponding collar assembly 330, 332. As the collar assemblies 330, 332 are caused to longitudinally slide in opposite directions, collar assembly-applied tensions in the cables 88, 90 are altered in a like fashion. In other words, the sliding collar assemblies 330, 332 simultaneously increase tension in the first cable 88 and decreased tension in the second cable 90, or vice-versa, to thus change the articulation arrangement as previously described. As a point of reference, in the view of FIG. 10B, the deflection assembly 58 is shown in an articulated or curved arrangement as otherwise prompted by increased tension in the first cable 88 (and thus rearward movement of the first collar assembly 330). It will be understood that for purposes of explanation and illustration, the first collar assembly 330 is shown in a more forward location (than might otherwise be sufficient to effectuate the illustrated articulation of the deflection assembly 58). That is to say, the relationship of the collar assemblies 330, 332 may not directly equate to the curve shown in the deflection assembly 58; rather, the articulated state of the deflection assembly 58 is shown in FIG. 10B merely to illustrate a possible radius of curvature available with the articulation mechanism 314.

The end effector rotation mechanism 316 is akin to the end effector rotation mechanism 116 (FIG. 5) described above, and includes an actuator 376 connected to the rod 320. In some constructions, the actuator 376 is a control knob that is rotatably assembled to the handle body 310 and directly connected to the rod 320. Other configurations are also envisioned, and in some embodiments, one or more gears can rotatably link the rod 320 and the actuator knob 376. Regardless, rotation of the end effector rotation actuator 376 is transferred to the end effector 52 via the rod 320, and causes rotation of the end effector 52 relative to the shaft 54. Further, the end effector rotation mechanism 316 does not impede operation of the end effector operation mechanism 312, permitting the rod 320 to move longitudinally as described above (e.g., where the actuator knob 376 is directly coupled to the rod 320, the actuator knob 376/handle body 310 mounting permits the actuator knob 376 to move longitudinally).

The optional shaft rotation mechanism 318 includes, in some embodiments, a shaft rotation actuator 380 rotatably maintained by the handle body 310 and assembled to the shaft 54. As with previous embodiments, the shaft rotation actuator 380 can be a knob forming a contoured outer surface 382 defining one or more grooves 384 adapted for convenient grasping or manipulation by a user. The shaft rotation knob 380 further forms an internal bore 386 sized and shaped in accordance with the first collar member 340, 344 of the collar assemblies 330, 332 (e.g., the first collar members 340, 344 and the bore 386 have a square perimeter). With this construction, and as described above, rotation of the shaft rotation knob 380 is transferred to the first collar members 340, 344, and in turn the cables 88, 90. Thus, as the shaft 54 is rotated with movement of the shaft rotation knob 380, the cables 88, 90 rotate in tandem, maintaining the effectuated bend at the deflection assembly 58, and preventing binding or twisting of the cables 88, 90.

While the collar assemblies 330, 332 are rotationally locked relative to the shaft rotation knob 380 via the bore 386, the collar assemblies 330, 332 can freely slide (longitudinally) such that the shaft rotation mechanism 318 does not impede operation of the articulation mechanism 314. Further, rotation of the shaft rotation knob 380 is mechanically isolated from the rod 320. Finally, an arrangement of the bore 386 relative to the collar assemblies 330, 332 and the linkage 334 permits rotation of the first collar members 330, 344 with rotation of the knob 380 without interference from the linkage 334 (and in particular the drive arms 350, 352). For example, the bore 386 has stepped regions that slidably capture the first collar members 330, 344 and provide clearance over the drive arms 350, 352 (and/or the drive arms 350, 352 are arranged to be “within” a perimeter of the first collar members 330, 344). In the position of the second collar assembly 332 of FIG. 10B, the second drive arms 352 is outside of the knob 380 and thus will not rotate with rotation of the knob 380. With this relationship, the drive arms 350, 352 do not interfere with desired rotation of the knob 380.

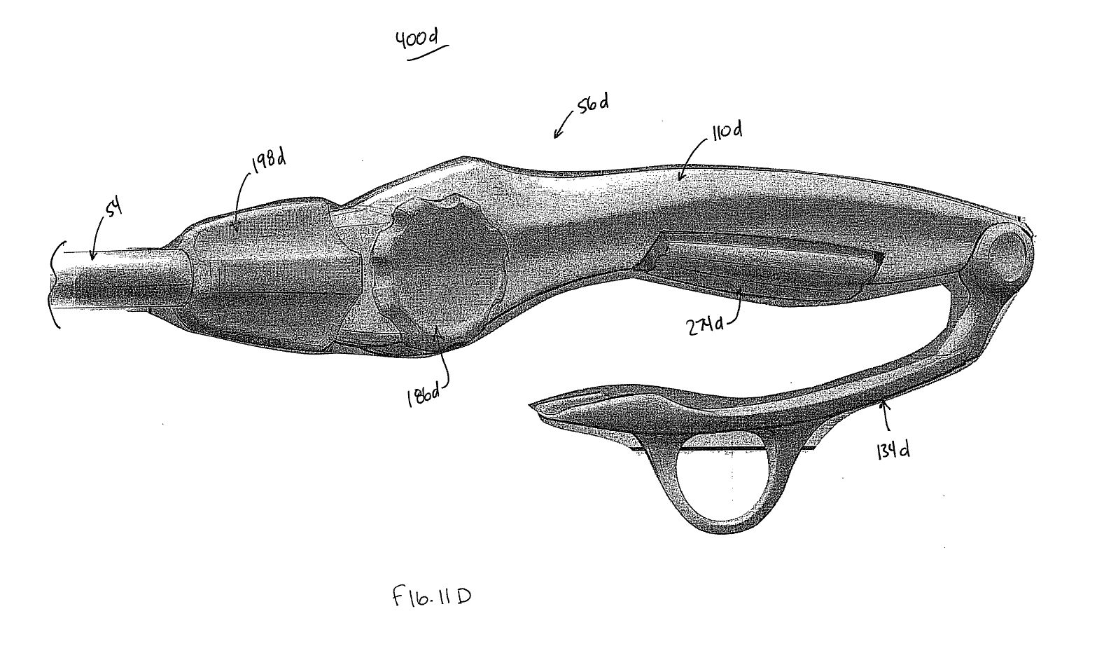

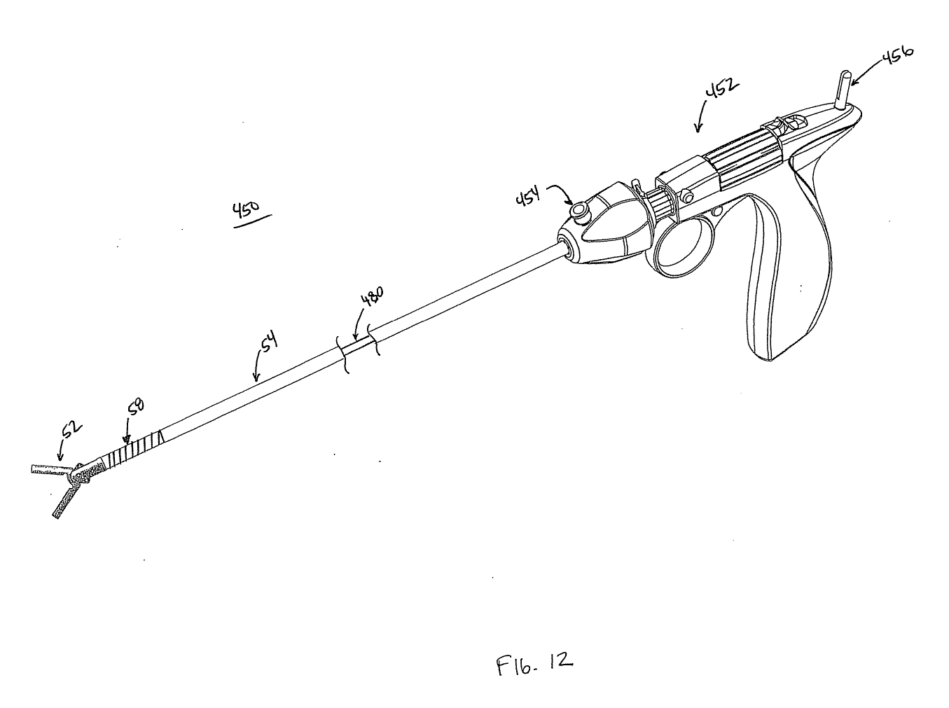

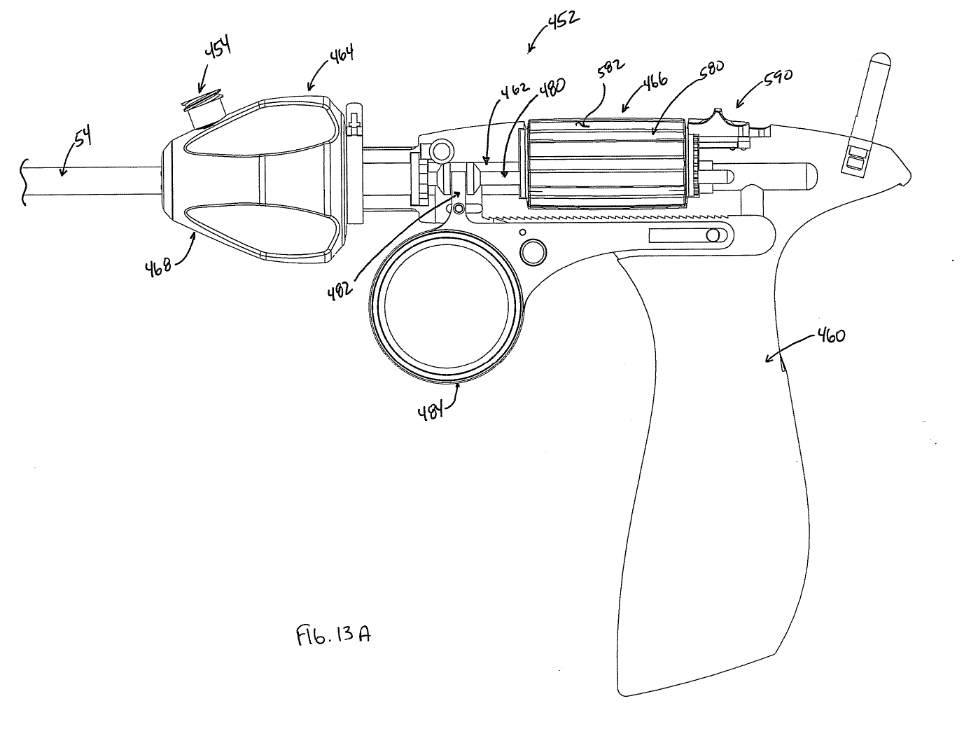

The sliding collar configuration associated with the instruments 50, 300 described above can be incorporated with various other handle assembly constructions in accordance with principles of the present disclosure. For example, the handle body and end effector operation mechanism can assume various other formats, each conducive to different, desired user handling of the instrument. In this regard, FIGS. 11A-11D illustrate a portion of other alternative articulating laparoscopic surgical instruments 400a-400d in accordance with principles of the present disclosure. With each of the instruments 400a-400d, a handle assembly 56a-56d is provided, and includes a handle body 110a-110d. Though primarily hidden in each of the views, the end effector operation mechanism, articulation mechanism, end effector rotation mechanism, and shaft rotation mechanism described above are also included, with the end effector operation mechanism associated with each instrument 400a-400d having the sliding collar construction as described above. The views of FIGS. 11A-11D illustrate a relationship of the various mechanism actuators relative to the corresponding handle body 110a-110d.

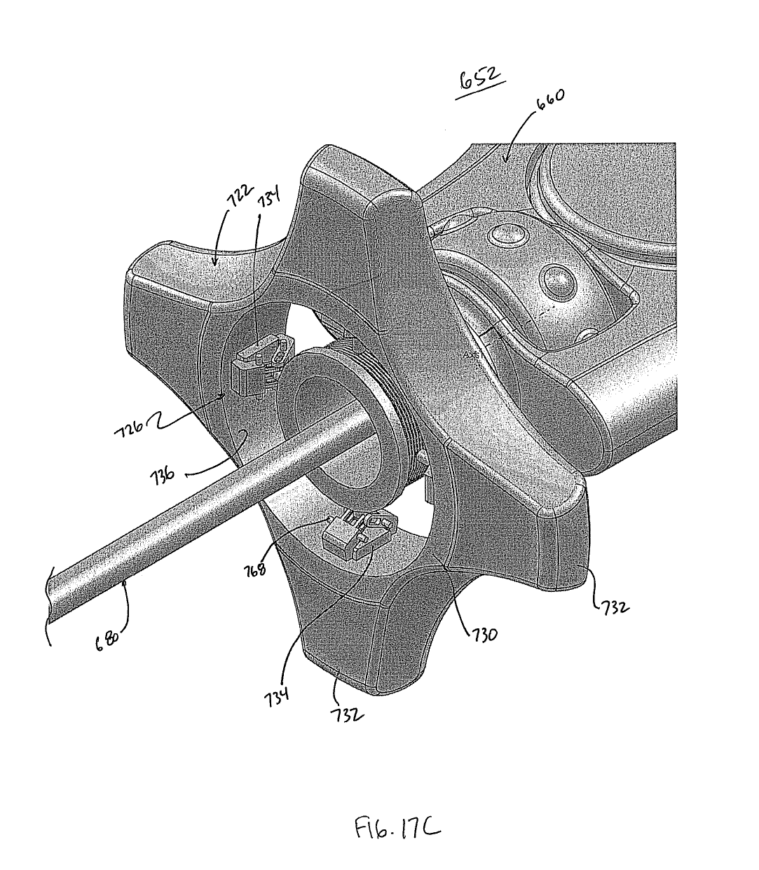

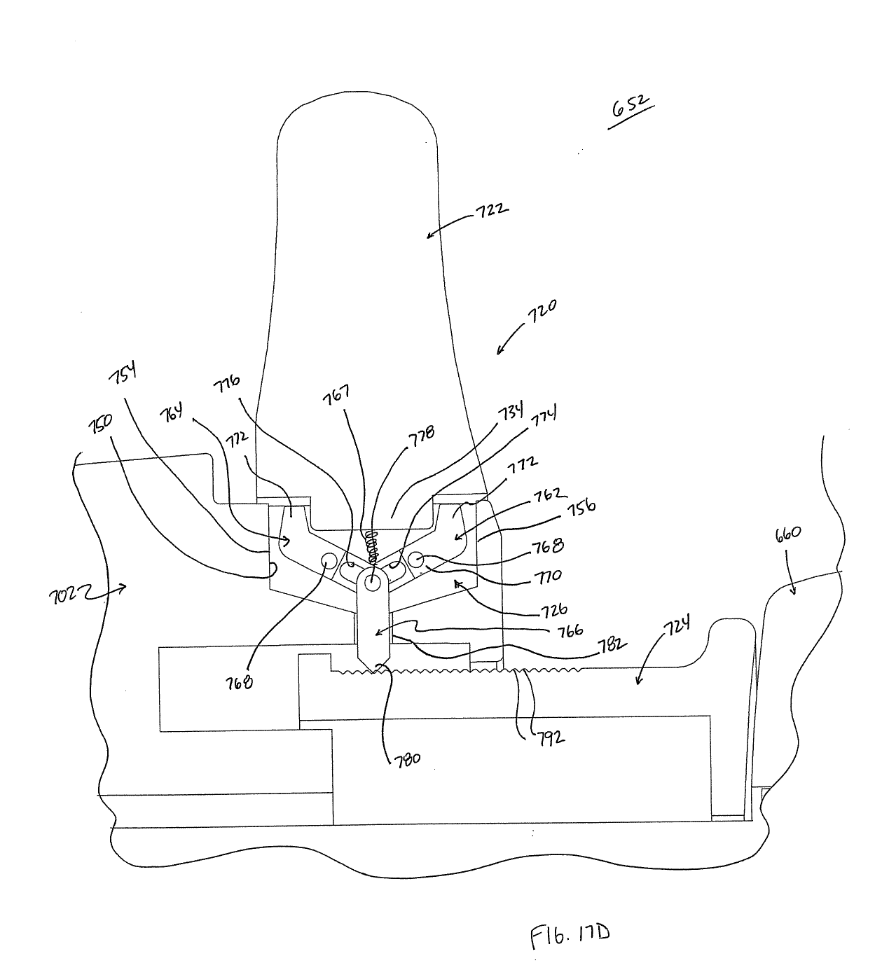

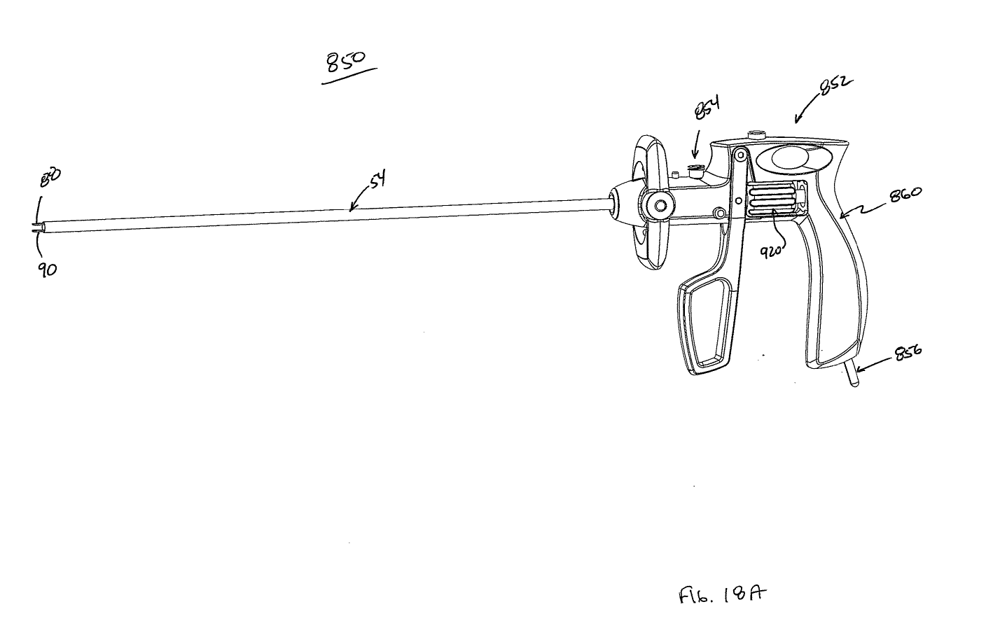

For example, with the handle assembly 56a of FIG. 11A, an end effector operation actuator 134a is pivotably coupled to the handle body 110a, forming a pistol grip-like construction. An articulation actuator 186a is provided as one or more levers along an exterior of the handle body 110a. User-caused movement of the lever 186a effectuates articulation/straightening of the deflection assembly as previously described. A locking device 402 (referenced generally) operates to selectively lock the lever 186a relative to the handle body 110a, and thus the deflection assembly 58 (FIG. 1) in a desired articulation arrangement. An end effector rotation actuator 274a is rotatably maintained by the handle body 110a, and is akin to a thumb wheel. Finally, a shaft rotation actuator 198a (in the form of a knob) is rotatably coupled to the handle body 110a, and is operable to rotate the shaft 54 consistent with previous explanations. Optionally, an outer collar 403 is provided and is rotatably linked to the knob 198a. Connection between the collar 403 and the knob 158a is described in greater detail below with respect to the constructions of FIGS. 17A-17D. In general terms, the collar 403 can be rotated slightly relative to the handle body 110a before rotationally engaging the knob 198a. Once engaged, continued rotation of the collar 403 is transferred onto the knob 198a, and thus onto the shaft 54. Additional components carried by the collar 403 selectively lock the collar 403/knob 198a at a selected rotational position.

FIG. 11A illustrates an optional flush port assembly 60a associated with the knob 198a and the shaft 54. Additionally, the handle assembly 56a of FIG. 11A, as well as any other of the handle assemblies of the present disclosure, can optionally include a conventional cautery post 404 (e.g., a mono-polar cautery post) mounted to the handle body 110a.

The handle assembly 56b of FIG. 11B is highly similar to the handle assembly 56a (FIG. 11A) described above. An articulation actuator 186b (i.e., lever) is configured (and operates) as above, as is an actuator 134b, an end effector rotation actuator 274b (i.e., thumb wheel) and a shaft rotation actuator 198b (i.e., knob). An end effector operation actuator 134b is also included, and is linked to an internal push/pull rod (not shown). With the construction of FIG. 11B, however, a pivotable coupling 406 of the end effector operation actuator 134b to the handle body 110b is further spaced from the rod (as compared to the previous embodiments), providing an enhanced mechanical advantage for user-applied forces.

With the handle assembly 56c of FIG. 11C, an end effector operation actuator 134c is pivotably coupled to the handle body 110a, with the components 110c, 134c combining to define an in-line style handle assembly (as compared to the pistol-grip styled handle assemblies described above). An articulation actuator 186c, an end effector rotation actuator 274c, and a shaft rotation actuator 198c are also shown. With the embodiment of FIG. 11C, the articulation actuator 186c is a thumb wheel-type component rotatably maintained by the handle body 110c. The end effector rotation actuator 274c also has a thumb wheel-like construction, and is accessible at an underside of the handle body 110c. Finally, the shaft rotation actuator 198c is provided as a rotatable knob at an end of the handle body 110c.

The handle assembly 56d of FIG. 11D is highly similar to the handle assembly 56c (FIG. 11C) described above, with the Figure illustrating various mechanism actuators relative to the handle body 110d. In particular, an end effector operation actuator 134d, an articulation actuator 186d, an end effector rotation actuator 274d, and a shaft rotation actuator 198d are provided. Each of the actuators, and corresponding mechanisms, are akin to the descriptions provided above. With the construction of FIG. 11D, however, the end effector operation actuator 134d is pivotably mounted at an end of the handle body 110d, with the components 110d, 134d combining to define an in-line type grip and enhanced mechanical advantage for a user-applied squeezing force.

Handle Assembly with Dual Function Control Knob

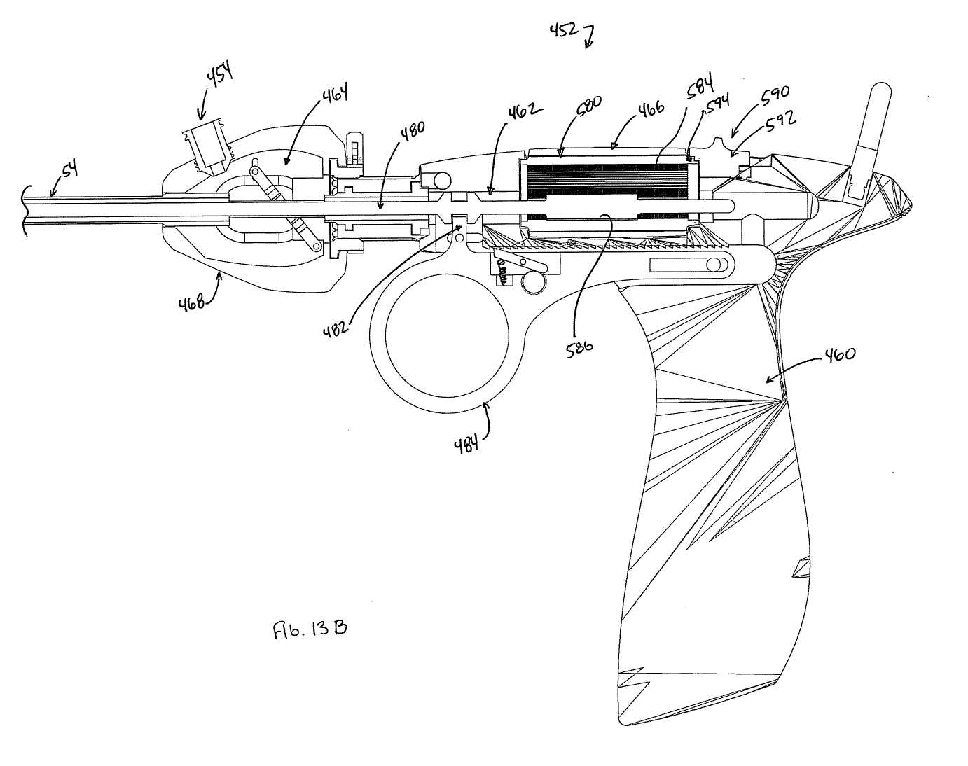

Yet another embodiment articulating laparoscopic surgical instrument 450 is shown in FIG. 12. In many respects the instrument 450 is akin to previous descriptions, and includes the end effector 52 and the shaft 54 as described above, with the shaft 54 forming or connected to the deflection assembly 58 in accordance with any of the previous or foregoing descriptions. In addition, the instrument 450 includes a handle assembly 452 that includes components of various mechanisms employed by a user during operation of the instrument 450. For example, and as described below, the handle assembly 452 incorporates articulation and shaft rotation mechanisms operable by a common actuator. Further, the instrument 450 optionally includes a flush port assembly 454 that facilitates cleaning and sterilization of the inner components of the instrument 450 and/or a cautery post 456 commensurate with previous descriptions.

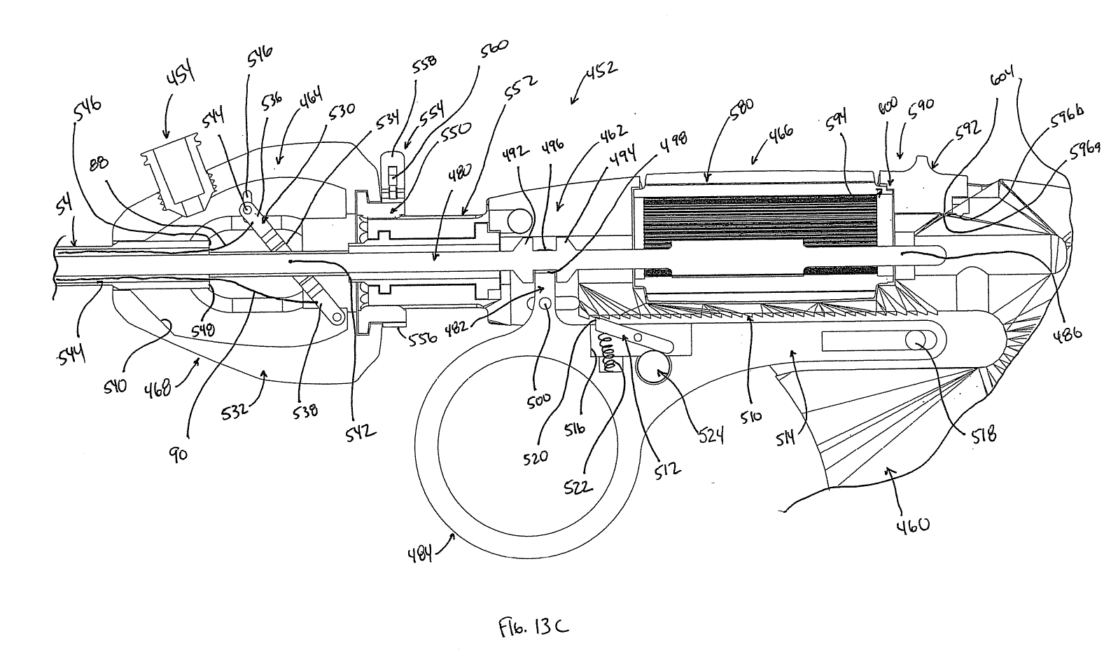

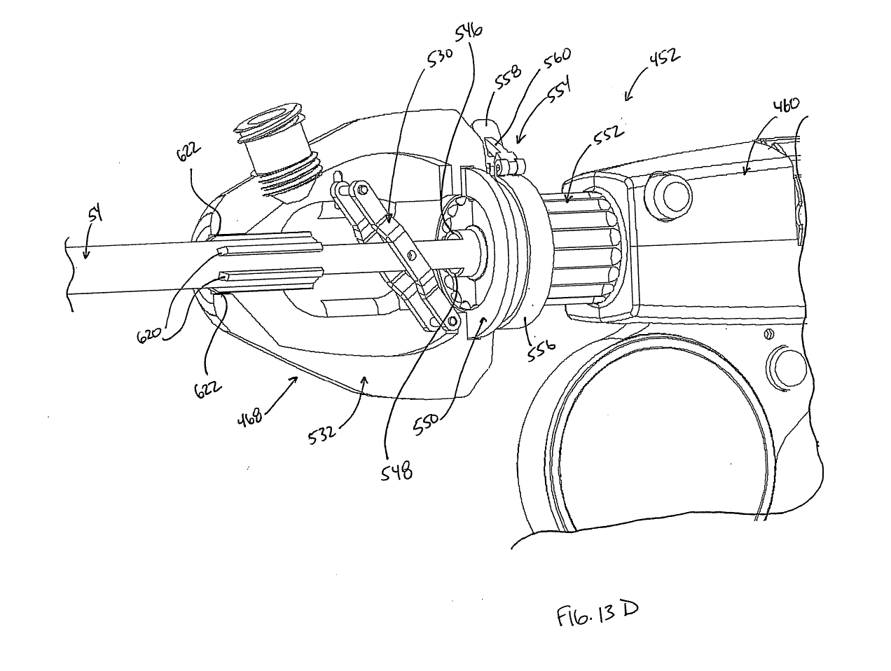

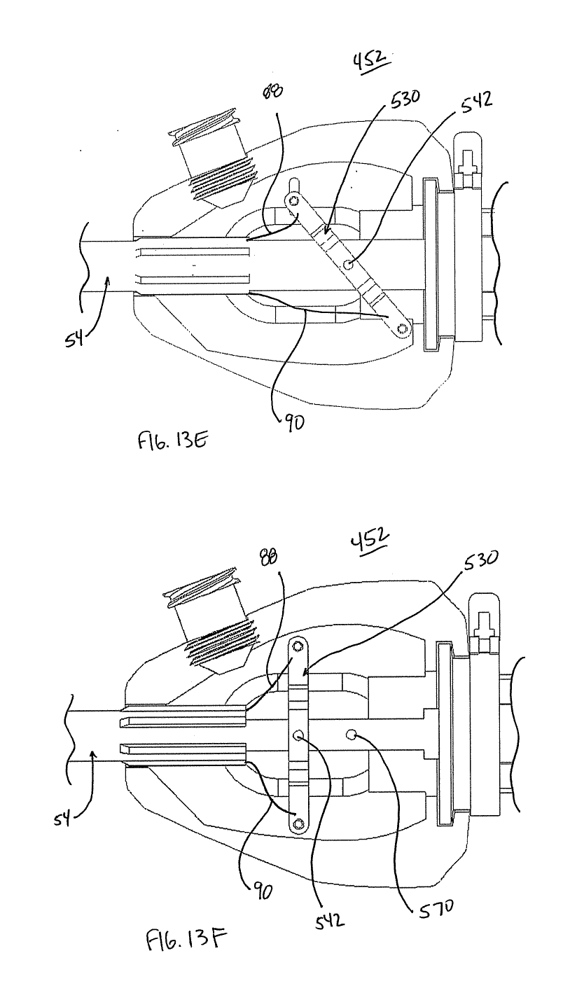

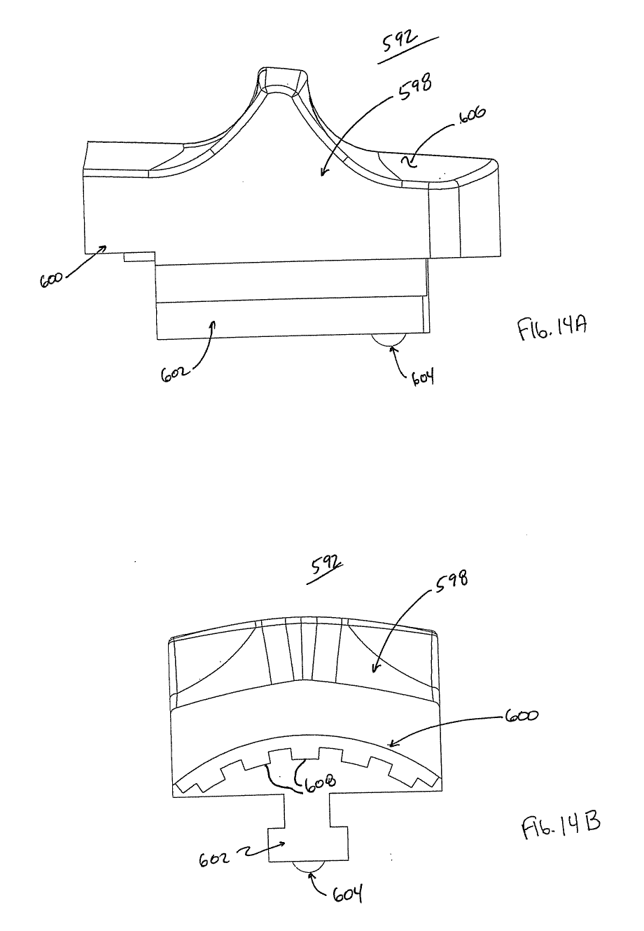

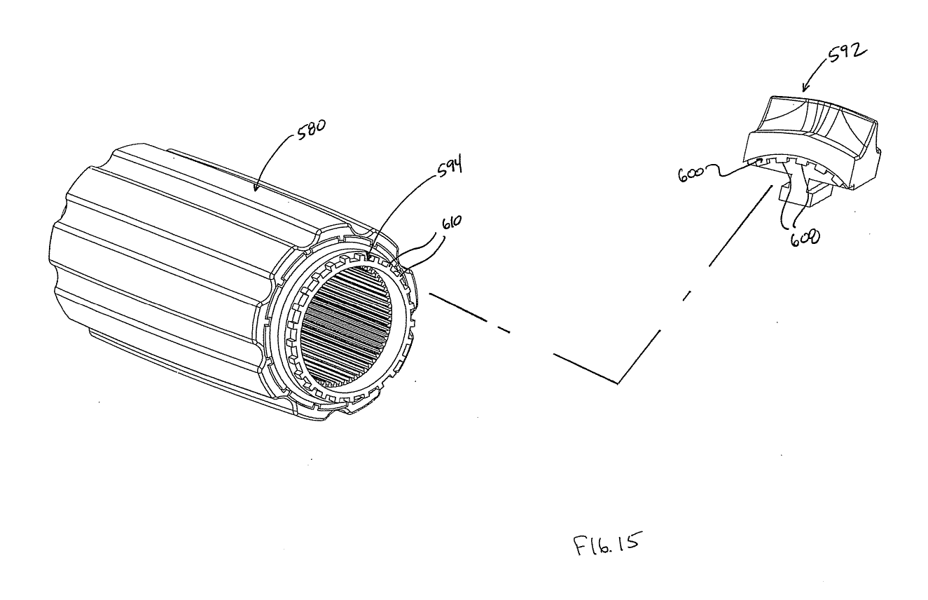

The handle assembly 452 is shown in greater detail in FIGS. 13A and 13B, and generally includes a handle body 460, an end effector operation mechanism 462, an articulation mechanism 464, an end effector rotation mechanism 466, and an optional shaft rotation mechanism 468. Each of the components are described in greater detail below. In general terms, however, as with previous embodiments the end effector operation mechanism 462 provides user control over an operative arrangement (e.g., opening/closing) of the end effector 52 (FIG. 12); the articulation mechanism 464 provides user control over articulation/straightening of the deflection assembly 58 (FIG. 12); the end effector rotation mechanism 466 provides user control over a rotational orientation of the end effector 52 relative to the shaft 54; and the shaft rotation mechanism 468 provides user control over a rotational position of the shaft 54 relative to the handle body 460.

The handle body 460 can assume any of the forms implicated by the present disclosure. In general terms, the handle body 460 is sized and shaped for convenient handling by a user (e.g., single-handed grasping) and maintains various components of the mechanisms 462-468 as described below.

The end effector operation mechanism 462 includes a rod 480, a slide body 482, and an end effector operation actuator 484. The rod 480 is akin to the rod 130 (FIG. 5), and can assume any of the forms previously described. The rod 480 includes or defines a proximal portion 486 slidably disposed within the handle body 460, and extends through the shaft 54. Though not shown, a distal portion of the rod 480 is coupled to the end effector 52 (FIG. 12) such that longitudinal movement (push/pull) of the rod 480 relative to the end effector 52/shaft 54 causes a change in an operative arrangement of the end effector 52.

The slide body 482 establishes a coupling between the rod 480 and the actuator 484, and is integrally formed by or with the actuator 484. A captured arrangement between the rod 480 and the slide body 482 is such that the rod 480 is caused to longitudinally slide with movement of the slide body 482, while permitting free rotation of the rod 480 relative to the slide body 482. For example and as better shown in FIG. 13C, first and second hubs 492, 494 are mounted to, or formed by, the rod 480. The slide body 482 defines a flange 496 having a width or thickness commensurate with (e.g., slightly less than) a spacing between the hubs 492, 494. Upon final assembly, the flange 496 is longitudinally captured between the hubs 492, 494 such that proximal or distal (longitudinal) movement of the slide body 482 is transferred to the rod 480 via the hub 492 or 494. The rod 480 is vertically retained by the handle body 460 slightly above a bearing surface 498 of the flange 496 so as to permit rotation of the rod 480 relative thereto. In some constructions, a vertical relationship of the slide body 482 relative to the rod 480 is maintained by a pin 500 that slidably couples the slide body 482 relative to the handle body 460.

The end effector operation actuator 484 can assume various forms, and in the illustrated embodiment has a trigger-like shape, combining with the handle body 460 to form the handle assembly 452 as a pistol grip. The trigger actuator 484 integrally forms, or is linked to, the slide body 482 such that user-caused movement of the actuator 484 relative to the handle body 460 is transferred onto the rod 480 via the slide body 482.

In some constructions, the end effector operation mechanism 462 further includes features for temporarily locking the trigger actuator 484 relative to the handle body 460, thus temporarily locking the rod 480 at a selected longitudinal position relative to the handle body 460 as dictated by the slide body 482/actuator 484. For example, the handle body 460 forms a toothed surface 510, and a ratchet arm 512 is associated with the trigger actuator 484. More particularly, a platform 514 extends from (or is integrally formed with) the trigger actuator 484, and forms a cavity 516 within which the ratchet arm 512 is pivotably maintained. The platform 514 is positioned relative to the toothed surface 510 (e.g., via a post 518) so that a leading end 520 of the ratchet arm 512 can be selectively brought into abutting engagement individual teeth of the toothed surface 510. A spring or other biasing member 522 is arranged to normally bias the leading end 520 into fixed or locked engagement with the toothed surface 510 as shown. In this regard, a shape of the teeth associated with the toothed surface 510, as well as a shape or angle of the leading end 520, is such that in the locked position, the toothed surface 510/leading end 520 interface impedes distal (leftward relative to the orientation of FIGS. 13A-13C) movement of the trigger actuator 484 relative to the handle body 460, but permits proximal (rightward) movement. Thus, in the locked position of FIGS. 13A-13C, an external force applied to the end effector 52 (FIG. 12) that would otherwise impart a pulling force onto the rod 480 (in the distal direction) will not result in longitudinal movement of the rod 480 (or a corresponding change in the selected operative arrangement of the end effector 52). Conversely, however, even in the locked position, a squeezing force applied by a user onto the trigger actuator 484 results in the leading end 520 sliding rearwardly along the toothed surface 510, and thus proximal longitudinal movement of the rod 480 via the slide body 482/hub 494 interface. A release button or cam-like body 524 is operatively associated with the ratchet arm 512 opposite the leading end 520. When selectively actuated by a user, the release button 524 applies a force onto the ratchet arm 512 sufficient to overcome a force of the spring 522, causing the ratchet arm 512 to pivot out of engagement with the toothed surface 510. Once released, the trigger actuator 484 can freely slide relative to the handle body 460.

The articulation mechanism 464 includes a pivot or articulation arm 530 and a knob 532. As described in greater detail below, with the embodiment of FIGS. 12-13C, the knob 532 serves as an actuator for both the articulation mechanism 464 and the shaft rotation mechanism 468. With this in mind, the pivot arm 530 includes a central section 534 and opposing end sections 536, 538. The central section 534 is pivotably coupled or pinned to the shaft 54 such that the end sections 536, 538 extend in opposite directions relative to the shaft 54. The knob 532 forms a cavity 540 sized and shaped to permit rotation of the pivot arm 530 about a pivot point 542 (identified generally in FIG. 13C) defined at the point of attachment between the central section 534 and the shaft 54. The first end section 536 is pivotably linked to the knob 532, whereas the second end section 538 is free of any direct coupling to the knob 532. In this regard, a pin 544 is rotatably secured to the first end section 536 and is slidably captured within a slot 546 in the knob 532. Thus, the first end section 536 can pivot and translate transversely relative to the knob 532 along a path of the slot 546. Longitudinal movement of the knob 532 is transferred to pivot arm 530 via the pinned interface 544/546, causing the pivot arm 530 to rotate about the pivot point 542, with the coupling between the first end section 536 and the knob 532 permitting the first end section 536 to translate transversely during rotation. The cables 88, 90 are mounted to a corresponding one of the end sections 536, 538. As best shown in FIG. 13C, the cables 88, 90 extend from the corresponding end section 536, 538, and into a lumen 544 of the shaft 54 via cut-outs 546, 548 (also identified in FIG. 13D) formed in the shaft 54. Commensurate with previous explanations, cables 88, 90 extend through the shaft 54 and are mounted to the deflection assembly 58 (FIG. 12).

The knob 532 can incorporate various features (e.g., contoured exterior surface) that promotes ease of user manipulation. Further, and as reflected in FIGS. 13A-13C, the optional flush port assembly 454 can be assembled to and pass through the knob 532. The knob 532 is slidably (longitudinally) connected with the handle body 460 in various manners. As identified in FIG. 13C, a sleeve 550 is interposed between the knob 532 and a nose 552 of the handle body 460. As described in greater detail below, the knob 532 is longitudinally fixed to sleeve 550, whereas the sleeve 550 can slide (longitudinally) over the nose 552. In other words, the knob 532 is longitudinally moveable relative to the handle body 460 via the sliding interface between the sleeve 550/nose 552. Other coupling configurations are also acceptable, such as slidably mounting the knob 532 directly to the nose 552. Regardless, for reasons made clear below, the knob 532 is longitudinally free of the shaft 54. Thus, the shaft 54 remains stationary with longitudinal movement of the knob 532.

With the above construction, longitudinal movement of the knob 532 relative to the handle body 460 and the shaft 54 causes the pivot arm 530 to rotate or pivot about the pivot point 542. This action, in turn, alters the tension in, or forces applied to, the cables 88, 90 in an equal but opposite manner. For example, relative to the orientation of FIG. 13C, proximal (or rightward) longitudinal movement of the knob 532 causes the pivot arm 530 to rotate clockwise due to the pinned coupling with the shaft 54 (it being recalled that the shaft 54 is longitudinally de-coupled from the knob 532). This rotation, in turn, increases tension in the first cable 88 (otherwise attached to the first end section 536) while simultaneously lessening a tension in the second cable 90 (otherwise attached to the second end section 538). A forced distal movement of the knob 532 relative to the handle body 460 effectuates an opposite change in cable tensions. The opposing change in tension or force applied to the cables 88, 90 in turn causes the deflection assembly 58 (FIG. 12) to articulate/bend or straighten as described above. Notably, however, longitudinal movement of the knob 532 is not directly transferred onto the rod 480. That is to say, the pivot arm 530, the knob 532, and the cables 88, 90 are mechanically isolated from the rod 480 such that operation of the articulation mechanism 464 does not alter a longitudinal position of the rod 480 relative to the handle body 460 (as otherwise dictated by the end effector operation mechanism 462), such that the deflection assembly 58 can be manipulated as desired without altering the selected operational arrangement of the end effector 52 (FIG. 12).

In some embodiments, a longitudinal position of the knob 532 relative to the handle body 460 can be controlled or “locked” by a clamp device 554. With reference to FIGS. 13C and 13D, the clamp device 554 includes a clamp 556 and complementary locking levers 558, 560. The clamp 556 is associated with the knob 532, and is transitionable between the loosened position shown and a tightened position. The sleeve 550, that otherwise functionally operates as part of the shaft rotation mechanism 468 described below, is disposed between the knob 532 and the nose 552 of the handle body 460 as mentioned above. The knob 532/sleeve 550 coupling permits rotation of the knob 532 relative to the sleeve 550, but prevents discrete longitudinal movement of the components 532, 550 relative to one another. In the tightened position, the clamp 556 frictionally locks the sleeve 550 over the nose 552 of the handle body 460. The locking levers 558, 560 are operable to releasably secure the clamp 556 in the tightened state, and can be disengaged by a user as desired. In the tightened state, the knob 532 is longitudinally locked relative to the handle body 460, such that the arrangement of the deflection assembly 58 (FIG. 12), as otherwise dictated by a longitudinal position of the knob 532, will not change.

Optionally, the handle assembly 452 can incorporate cleaning mode features in which a coupling between the pivot arm 530 and the shaft 54 is selectively releasable (e.g., a switch or similar device is provided that permits a user to readily disconnect and re-assemble the pivot arm 530 relative to the shaft 54 at the pivot point 542). FIGS. 13E and 13F illustrate one example of a cleaning mode configuration envisioned by the present disclosure. In particular, FIG. 13E depicts a portion of the handle assembly 452 described above in a use mode. The pivot arm 530 is pivotably coupled to the shaft 54 at the pivot point 542 by a releasable pin or similar device. In the cleaning mode of FIG. 13F, the pivot arm 530 is released from the direct coupling to the shaft 54 (e.g., the release pin is removed by a user), allowing the pivot arm 530 to freely move longitudinally relative to the shaft 54. As a point of reference, FIG. 13F identifies at 570 a location along the shaft 54 at which the pivot arm 530 is pivotably coupled thereto in the use mode; the pivot point 542 on the pivot arm 530 is also identified, it being understood, however, that in the cleaning mode, the pivot arm 530 is no longer coupled to the shaft 54. When the pivot arm 530 is released from the shaft 54 (i.e., cleaning mode), the knob 464/pivot arm 530 can be moved longitudinally forward relative to the shaft 54 to crate slack in the cables 88, 90. This slack, in turn, can facilitate separation of components of the deflection assembly 58 (FIG. 12) for cleaning purposes as described above. The releasable coupling configuration of FIGS. 13E and 13F can be employed with any of the instruments/handle assemblies of the present disclosure. In other embodiments, the cleaning mode feature is omitted.

With reference to FIGS. 13A and 13B, the end effector rotation mechanism 466 includes the rod 480 and an end effector rotation actuator 580 that is rotatably coupled to the handle body 460. The actuator 580 can be a tubular body, forming a contoured outer surface 582 (FIG. 13A). The contoured outer surface 582 provides a convenient surface (e.g., grooves or protrusions) for interface by a user's thumb or finger. As visible in FIG. 13B, a first gear 584 is formed by the actuator 580 (e.g., an internal barrel gear). The first gear 584 meshingly engages a second gear 586 formed or provided along the rod 480 in a planetary gear-like fashion. With this construction, rotation of the actuator 580 is transferred to the rod 480 via the first gear 584/second gear 586 interface, resulting in rotation of the end effector 52 (FIG. 12) relative to the shaft 54. Notably, the meshed interface between teeth of the first gear 584/second gear 586 is such that the rod 480 can slide or move longitudinally relative to the end effector rotation actuator 580 (e.g., with operation of the end effector operation mechanism 462) while maintaining the meshed engagement.

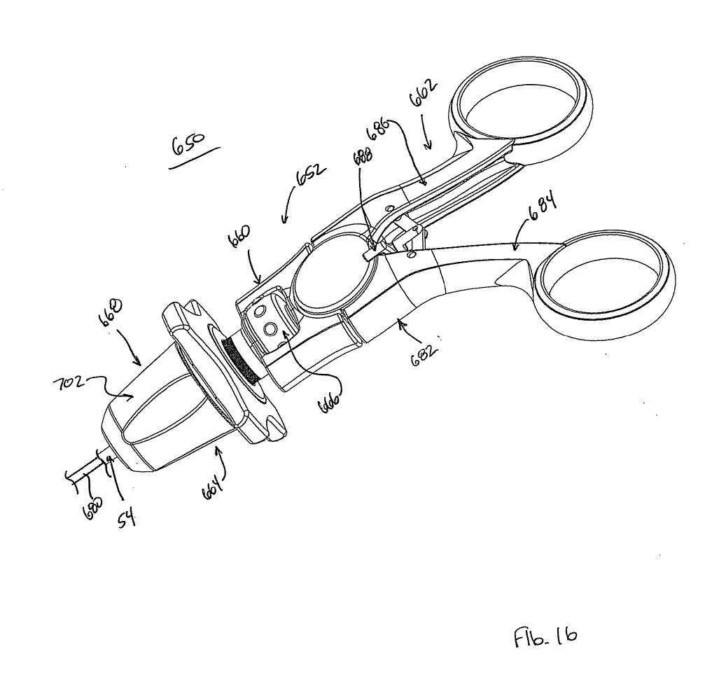

A rotational arrangement of the end effector rotation actuator 580 relative to the handle body 460 (and thus of the rod 480/end effector 52 relative to the handle body 460) is selectively fixed or locked by an optional locking device 590. The locking device 590 includes a switch 592 and a sprocket 594. As more clearly evident in FIG. 13C, the handle body 460 slidably retains the switch 592, and forms locking dimples 596a, 596b configured to interface with the switch 592 as described below. The switch 592 is shown in greater detail in FIGS. 14A and 14B, and forms or defines a switch body 598, an engagement shoulder 600, a foot 602, and a locking tab 604. The switch body 598 has a contoured outer surface 606 configured for convenient interface by a user's finger/thumb. The engagement shoulder 600 has an arcuate shape as best shown in FIG. 14B, and forms teeth 608. The foot 602 extends from the switch body 598 and is configured to be slidably captured by a correspondingly-sized slot in the handle body 460 (FIG. 13C). Finally, the tab 604 projects from the foot 602 and is sized to be received within respective ones of the dimples 596a, 596b (FIG. 13C).

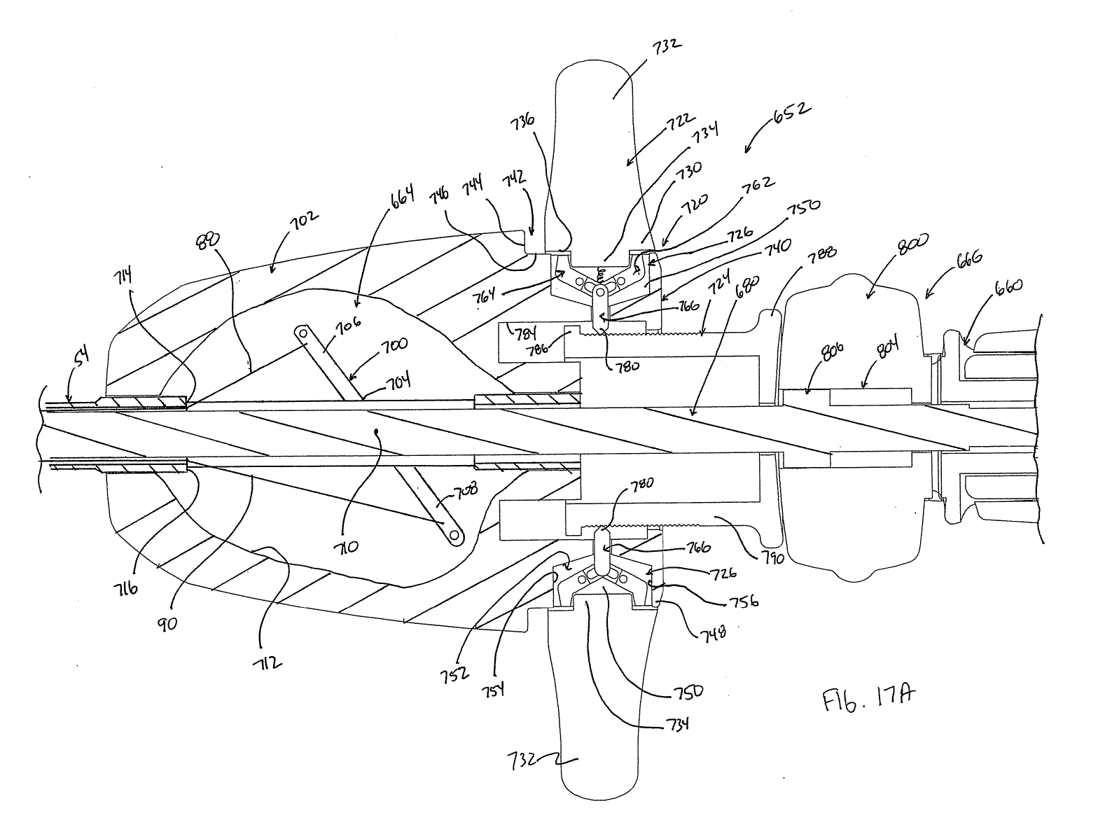

The sprocket 594 is shown in greater detail in FIG. 15 and is attached to or formed by the end effector rotation actuator 580. Teeth 610 of the sprocket 594 are circumferentially spaced from one another, and are sized and spaced to selectively mesh with the teeth 608 of the switch shoulder 600. With reference between FIGS. 13C and 15, in the forward position (FIG. 13C) of the switch 592, the teeth 608 of the shoulder 600 engage the teeth 610 of the sprocket 594. Because the switch 592 is rotationally secured to the handle body 460, then, in the forward position, the switch 592 prevents rotation of the actuator 580. Further, the tab 604 is disposed within the forward dimple 596a so as to prevent inadvertent displacement of the switch 592 from the forward position. Where rotation of the end effector 52 (FIG. 12) is desired, a user transitions the switch 592 to a released position in which the tab 604 is disposed within the rearward locking dimple 596b, holding the shoulder 600 away from the sprocket 594. An arrangement of the teeth 608 of the switch shoulder 600 with the teeth 610 and the sprocket 594 freely permits longitudinal sliding movement of the switch 592. As a result, the actuator 580 can freely rotate relative to the handle body 460, thereby effectuating rotation of the rod 480, and thus the end effector 52 attached thereto, as desired.

The shaft rotation mechanism 468 includes, in some embodiments, the knob 532 and the sleeve 550 as described above. The knob 532 is rotationally connected to the shaft 54, and is rotatably coupled over the sleeve 550. For example, as best shown in FIG. 13D, splines 620 are formed along the shaft 54, and are slidably received within corresponding grooves 622 (referenced generally) defined by the knob 532. The spline 620/groove 622 rotationally captures the shaft 54 relative to the knob 532, but permits the knob 532 to slide longitudinally relative to the shaft 54. With this construction, rotation of the knob 532 about the sleeve 550 causes the shaft 54 to rotate. Notably, the pivot arm 530 is rotationally fixed to the shaft 54/knob 532; because the cables 88, 90 (omitted from the view of FIG. 13D, but shown in FIG. 13C) are attached to the pivot arm 530, then, the cables 88, 90 rotate with rotation of the shaft 54/knob 532. Thus, the cables 88, 90 will not bind or twist during shaft rotation. Further, while the knob 532 can rotate relative to the sleeve 550, the components 532, 550 are longitudinally fixed relative to one another. Thus, and with continued reference to FIG. 13C, the end effector operation mechanism 462 does not interfere with operation of the shaft rotation mechanism 468, and vice-versa. That is to say, the knob 532 is rotatably and longitudinally movable relative to the handle body 460, thereby facilitating the desired shaft rotation or end effector operation action. Further, rotation of the knob 532 is mechanically isolated from the rod 480. Thus, the shaft 54 can be rotated without rotation of the rod 480/end effector 52.

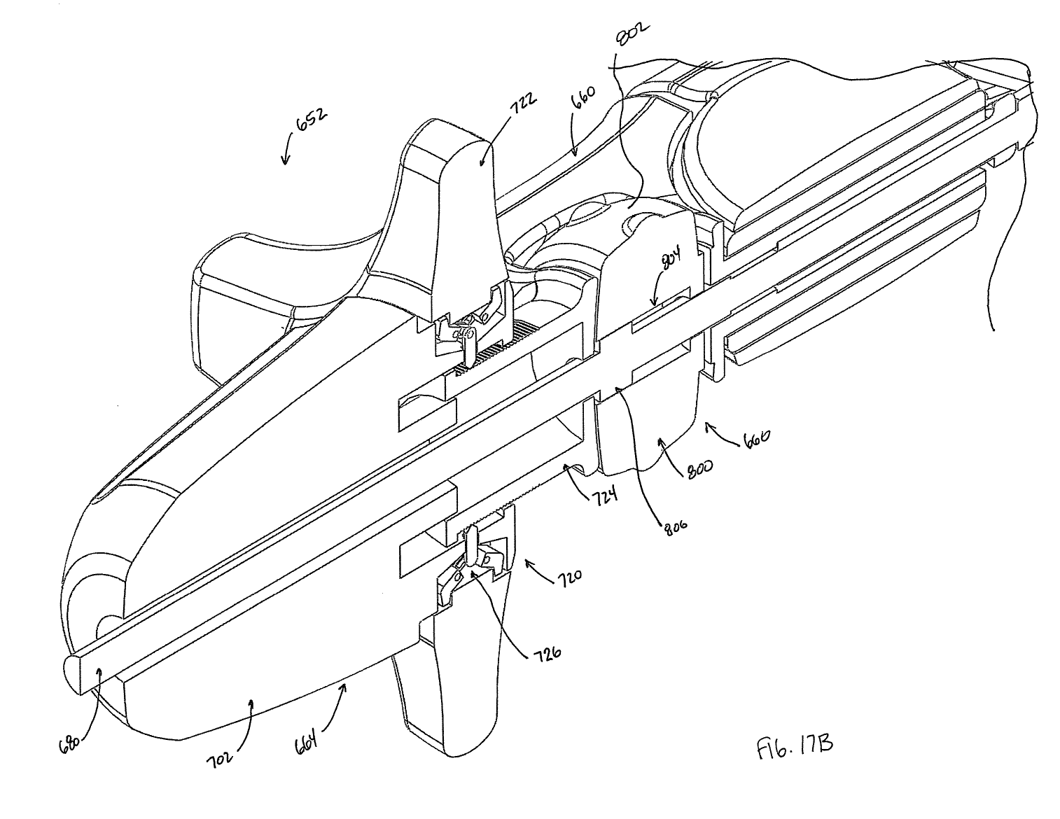

The dual function actuator knob construction of the instrument 450 can be incorporated with a variety of other handle assembly constructions. For example, a portion of a related embodiment articulating laparoscopic surgical instrument 650 in accordance with principles of the present disclosure is shown in FIG. 16. In many respects, the surgical instrument 650 is akin to the instrument 450 (FIG. 12) described above, and includes the end effector 52 (omitted from the view of FIG. 16, but shown in FIG. 1) and the shaft 54. A handle assembly 652 provides components of the various mechanisms described above. Although many of the internal components are hidden in the view of FIG. 16, the handle assembly 652 includes a handle body 660 maintaining or coupled to an end effector operation mechanism 662, an articulation mechanism 664, an end effector rotation mechanism 666, and a shaft rotation mechanism 668, with each of the mechanisms 662-668 being referenced generally in FIG. 16.

The end effector operation mechanism 662 includes a rod 680 (partially visible in the view of FIG. 16 and better shown in FIGS. 17A and 17B) and an end effector operation actuator 682. With the construction of FIG. 16, the actuator 682 includes opposing arms 684, 686 that are pivotably coupled to the handle body 660, and linked to the rod 680. The arms 684, 686 combine to define a scissors-like construction, rendering the handle assembly 652 to optionally have a more in-line shape as compared to previous embodiments. Alternatively, the pistol grip-like shape of previous embodiments is equally acceptable. Regardless, user-caused transverse movement of the arms 684, 686 toward or away from one another imparts a longitudinal force onto the rod 680, causing the rod 680 to move longitudinally, forward or rearward. Commensurate with previous descriptions, this action is, in turn, transferred to the end effector 52 (FIG. 1), resulting in a change in the end effector's 52 operational arrangement. An optional locking lever device 688 (referenced generally) can be provided that allows a user to selectively lock the actuator 682 (and thus the rod 680 and the end effector 52) at a desired longitudinal location of the rod 680. Further, connection between the actuator 682 and the rod 680 is such that the rod 680 can rotate with operation of the end effector rotation mechanism 666.

Components of the articulation mechanism 664 are better shown in FIGS. 17A and 17B, and include a pivot or articulation arm 700 and a dual function knob 702. For ease of illustration, components at an interior of the knob 702 (e.g., the pivot arm 700) are omitted from the view of FIG. 17B. In many respects, the articulation mechanism 664 is identical to the articulation mechanism 464 (FIG. 13C) described above. The pivot arm 700 forms or defines a central section 704 and opposing end sections 706, 708. The pivot arm 700 is pivotably coupled to the shaft 54 along the central section 704 to establish a pivot point 710. Further, the first end section 706 is pivotally coupled to the knob 702, with the knob 702 forming a cavity 712 sized to receive and permit rotation of the pivot arm 700 about the pivot point 710. Details of the knob 702/first end section 706 coupling are omitted from the views, but can be identical to the arrangement described above with respect to FIG. 13C. The first cable 88 is attached to and extends from the first end section 706, whereas the second cable 90 is attached to and extends from the second end section 708. The cables 88, 90 pass through a corresponding cut-out 714, 716 in the shaft 54, and are connected to the deflection assembly 58 (FIG. 1) in accordance with the above descriptions. The knob 702 is slidably mounted to the handle body 660 as described below. Finally, the knob 702 is longitudinally isolated from, but rotationally coupled to, the shaft 54 (e.g., similar to the construction of FIG. 13D).

With the above construction, distal or proximal longitudinal movement of the knob 702 relative to the handle body 660 is transferred to the pivot arm 700, causing the pivot arm 700 to rotate about the pivot point 710 (it being recalled that the pivot arm 700 is pinned to the shaft 54, and the shaft 54 remains stationary with longitudinal movement of the knob 702). This action, in turn, alters the applied force or tension in the cables 88, 90 in an opposing manner. This change in tension, in turn, causes the deflection assembly 58 (FIG. 1) to correspondingly articulate or straighten. As with previous embodiments, the articulation mechanism 664 is mechanically isolated from the rod 680 such that user-initiated movement of the knob 702 is not transferred onto the rod 680.

In some constructions, the articulation mechanism 664 includes a locking device 720 (referenced generally) that functions to selectively lock the knob 702 at a desired longitudinal location relative to the handle body 660 (and thus temporarily lock the deflection assembly 58 (FIG. 1) at a desired articulation arrangement). The locking device 720 includes a collar 722, a sleeve 724, and a plurality of engagement assemblies 726. The collar 722 is mounted to the knob 702, and the engagement assemblies 726 interfacing with the sleeve 724 in a manner that selectively longitudinally locks the knob 702/collar 722 relative to the sleeve 724.

As best shown in FIG. 17C (that otherwise illustrates a portion of the handle assembly 652 with the knob 702 (FIG. 16) removed), the collar 722 includes or forms a base 730 and several shoulders 732. The base 730 has a ring-like shape, sized for mounting to the knob 702 as described below. The shoulders 732 project radially outwardly from the base 730, and provide convenient surfaces for grasping by a user. While the collar 722 is shown as having four of the shoulders 732, any other number, either greater or lesser, is also acceptable. A plurality of circumferentially spaced support blocks 734 are formed or provided along an inner surface 736 of the base 730. The support blocks 734 can be aligned with respective ones of the shoulders 734, or can be off-set relative thereto. More or less than four of the support blocks 734 can be included. The support blocks 734 can be integrally formed by the collar 722 or can be separately formed and subsequently assembled to the inner surface 736. Regardless, each of the support blocks 734 retains or supports components of a corresponding one of the engagement assemblies 726 as described below.

Returning to FIG. 17A, a trailing portion 740 of the knob 702 is generally configured to receive the collar 722 such that the collar 722 cannot overtly rotate relative to the knob 702 and is longitudinally linked to the knob 702 (i.e., the collar 722 can slide longitudinally a short distance relative to the knob 702, but at an established longitudinal position between the components 702, 722, a longitudinal force on the collar 722 is transferred onto the knob 702). In particular, a shelf 742 is formed in the knob 702, defined by a radial ridge 744 and a circumferential ledge 746. A diameter of the ledge 746 is slightly less than an inner diameter of the collar base 730 (along the inner surface 736), such that the inner surface 736 can longitudinally slide along the ledge 746. Conversely, the ridge 744 defines an outer diameter greater than the diameter of the inner surface 736, and effectively serves as a stop to forward sliding movement of the collar 722 relative to the knob 702. As shown, the knob 702 can further form an annular foot 748 opposite the ledge 746 that is similarly sized and shaped to slidably support the inner surface 736 of the collar 722.

The trailing portion 740 of the knob 702 also forms or defines a series of radially spaced recesses 750 (two of which are visible in the view of FIG. 17A). The number and shape of the recesses 750 corresponds with the number and shape of the engagement assemblies 726/support blocks 734. In general terms, respective ones of the engagement assemblies 726/support blocks 734 are received within a corresponding one of the recesses 750, with a circumferential width of each of the recesses 750 corresponding with a circumferential width of each of the support blocks 734. With this construction, a rotational force applied to the collar 722 is transferred onto the knob 702 via abutting interface between the blocks 734 and a corresponding axial wall (one of which is identified at 752 in FIG. 17A) associated with the respective recess 750. A combined longitudinal length of the support block 734/engagement assembly 726 is less than a longitudinal length of each of the recesses 750 (with the recess length being defined between opposing radial walls 754, 756), permitting partial longitudinal sliding of the collar 722 relative to the knob 702.

As better illustrated in FIG. 17D, each of the engagement assemblies 726 includes opposing lever arms 762, 764, a pawl 766, and a biasing member (e.g., a spring) 767. The lever arms 762, 764 are pivotably coupled to the knob 702 (within the corresponding recess 750) at opposite sides, respectively, of the corresponding support block 734. For example, FIG. 17C illustrates dowels 768 projecting from each of the lever arms 762, 764. The dowels 768 are also identified in FIG. 17D and are rotatably received within holes (not shown) formed by the knob 702. The dowels 768 thus serve as a pivot point for the corresponding lever arm 762, 764. Each of the lever arms 762, 764 has an L-like shape. With respect to the first lever arm 762, leading and trailing segments 770, 772 extend from one another in an angled fashion, with the trailing segment 772 arranged to selectively bear against or abut the support block 734. More particularly, in the absence of external forces, an interface between the support block 734 and the lever arms 762, 764 naturally direct the lever arms 762, 764 to the spatial orientation shown. The leading segment 770 forms a slot 774. The second lever arm 764 similarly forms a slot 776.

The pawl 766 is coupled to each of the lever arms 762, 764 via a pin 778 slidably captured within the slots 774, 776. The pawl 766 terminates at an engagement end 780 opposite the lever arms 762, 764. As shown, the engagement end 780 can have a tapered shape appropriate for engagement with features provided on the sleeve 724 as described below. The knob 702 forms a hole 782 from the corresponding recess 750 through which the pawl 766 slidably passes.

The biasing member 767 is disposed between the support block 734 and the pawl 766. With this arrangement, the biasing member 767 forces the pawl 766 away from the support block 734 in the normal state reflected by FIG. 17D.

Upon final assembly, the engagement assemblies are transitionable between the natural lowered or locked state shown in FIG. 17A and a raised or released state via pivoting of the lever arms 762, 764 as described below. In the lowered state, the pawls 766 are directed onto engagement with the sleeve 724; in the raised state, the pawls 766 are radially displaced from the sleeve 724. Thus, a diameter collectively defined by the pawl engagement ends 780 in the lowered state is less than a collective diameter in the raised state.

The sleeve 724 is fixed to, or integrally formed by, the handle body 660. Further, the sleeve 724 is sized to be slidably disposed within an axial bore 784 formed in the knob 702, and defines a leading flange 786, a trailing flange 788, and an intermediate section 790. The leading flange 786 defines an outer diameter that is less than an inner diameter collectively defined by the pawls 766 (in either the lowered or raised states). Thus, the leading flange 786 serves as a stop to forward longitudinal movement of the knob 702 via abutting contact between the pawls 766 and the leading flange 786. The trailing flange 788 provides a similar stop to rearward movement of the knob 702 relative to the sleeve 724. The intermediate section 790 forms or defines a plurality of circumferential ribs or ridges 792 most clearly shown in FIG. 17D. A diameter of each of the ribs 792 is greater than the diameter collectively defined by the pawl engagement ends 780 in the lowered or locked state of the engagement assemblies 726 as shown. In the lowered state, the engagement end 780 of each of the pawls 766 is held between adjacent ones of the ribs 792, creating a longitudinal fixation between the pawls 766 and the sleeve 724. The sleeve 724 is longitudinally fixed to the handle body 660, whereas the pawls 766 are effectively longitudinally fixed relative to the knob 702. In the lowered or locked state of FIG. 17A, then, the engagement assemblies 726 naturally operate to prevent longitudinal movement of the knob 702 relative to the handle body 660 (and resist external forces applied at the deflection assembly 58 (FIG. 1) that would otherwise cause the deflection assembly 58 to deviate from a selected articulation arrangement). However, the engagement assemblies 726, and thus the collar 722/knob 702, can rotate relative to the handle body 660/sleeve 724 in the lowered state, with the pawl engagement ends 780 circumferentially sliding between the corresponding ribs 792.

Longitudinal movement of the knob 702 relative to the handle body 660 (and thus operation of the articulation mechanism 664 as described above) entails transitioning of the engagement assemblies 726 from the lowered state to the raised state. A user applies a pushing or pulling force (longitudinally directed) onto one or more of the shoulders 732. The collar 722 is thus caused to longitudinally slide relative to the knob 702 in a direction of the applied force. With sufficient movement, the support block 734 is brought into full contact with the corresponding one of the lever arms 762, 764, causing the lever arm 762, 764 to pivot and lift the pawl 766 away from the sleeve 724 once a force sufficient to overcome a force of the biasing member 767 is generated. For example, with specific reference to FIG. 17D, rearward (or rightward) longitudinal movement of the collar 722/support block 734 imparts a force onto the trailing segment 772 of the first lever arm 762, causing the first lever arm 762 to apply a force onto the biasing member 767 via the pawl 766, and then pivot about the pivot point 768, lifting the pawl 766 from the sleeve 724 to the raised or released state in which the knob 702 is no longer longitudinally fixed to the sleeve 724. Because the pawl 766 is linked to both of the lever arms 762, 764 via the pin 778, the second lever arm 764 is thus also caused to pivot, with the pin 778 sliding within each of the slots 774, 746. With further rearward movement of the collar 722, the trailing segment 772 wedges between the support block 734 and the recess trailing wall 756, such that the rearward movement or force of the collar 722/support block 734 is transferred onto the knob 702, causing the knob 702 to now slide in the same direction relative to the sleeve 724 (and thus relative to the handle body 660). Because the pawl 766 has been and remains lifted away from the sleeve 724, the locking device 720 no longer prevents longitudinal movement of the knob 702 relative to the sleeve 724/handle body 660.

Once a desired longitudinal position of the knob 702 relative to the handle body 660/sleeve 724 has been achieved (i.e., desired articulation of the deflection assembly 58 (FIG. 1)), the user-applied force onto the collar 722 is removed. The biasing member 767 naturally directs the pawl 766 toward the sleeve 724, with the lever arms 762, 764 caused to rotate or pivot back toward the lowered or locked state. The pawl 766 is thus returned into engagement with the sleeve 724 as described above. It will be understood that forward movement of the knob 702 relative to the handle body 660/sleeve 724 is facilitated in a similar manner, with forward movement of the collar 722 being transferred onto the second lever arm 764 and the recess leading wall 754, again causing the pawl 766 to be lifted from engagement with the sleeve 724.

Returning to FIGS. 17A and 17B, the end effector rotation mechanism 666 includes an end effector rotation actuator 800 and the rod 680. The actuator 800 is rotatably coupled to the handle body 660, and forms or defines a contoured outer surface 802. The contoured outer surface 802 can assume various shapes and configurations appropriate for convenient user interaction, and can be akin to a thumb wheel. A first gear 804 is formed by, or attached to, the thumb wheel 800 (e.g., the first gear 804 is an interior barrel gear surface by the actuator 800). The first gear 804 is configured to meshingly engage a second gear 806 formed by or assembled to the rod 680. Rotation of the actuator 800 is thus transferred onto the rod 680 (and thus to the end effector 52 (FIG. 1)) via the first gear 804/second gear 806 interface. As with previous embodiments, the first gear 804/second gear 806 interface is further configured to permit longitudinal movement of the rod 680 relative to the actuator 800, for example by the second gear 806 longitudinally sliding along the first gear 804. Thus, the end effector rotation mechanism 666 permits or facilitates operation of the end effector operation mechanism 662, and vice-versa.

The shaft rotation mechanism 668 includes the knob 702 that effectively serves as a dual function component, providing a user-operable actuator for both the articulation mechanism 664 and the shaft rotation mechanism 668. With respect to the shaft rotation mechanism 668, the knob 702 is rotationally fixed to the shaft 54 such that rotation of the knob 702 is transferred to the shaft 54 (while permitting longitudinal movement of the knob 702 relative to the shaft 54). Though hidden in the views, coupling between the knob 702 and the shaft 54 can assume the format described above with reference to FIG. 13D. Rotation of the shaft 54 can also be accomplished by rotation of the collar 722. As described above, the support blocks 734 provided with the collar 722 are circumferentially captured by the knob 702; rotation of the collar 722 is transferred to the knob 702 and thus to the shaft 54. Regardless, the pivot arm 700 is coupled to the knob 702 such that the pivot arm 700, and thus the cables 88, 90 mounted thereto, rotate with rotation of the knob 702/shaft 54. As a result, the cables 88, 90 will not bind or twist during rotation of the shaft 54. Though not expressly shown, the shaft rotation mechanism 668 can optionally further include components that selectively lock a rotational position of the shaft 54 relative to the handle body 660.

Handle Assembly with External Pivot Arm

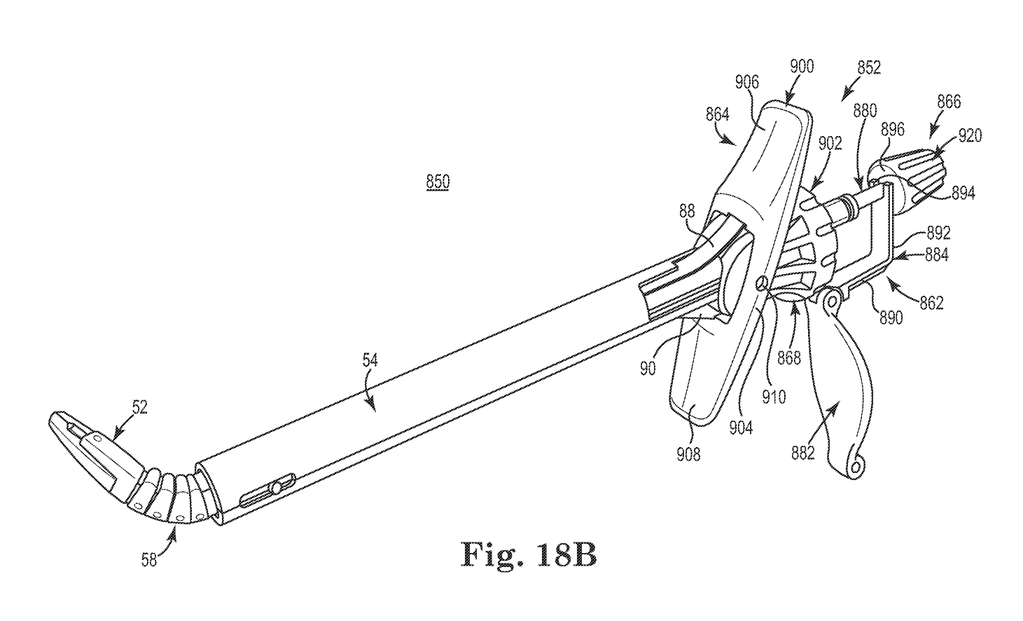

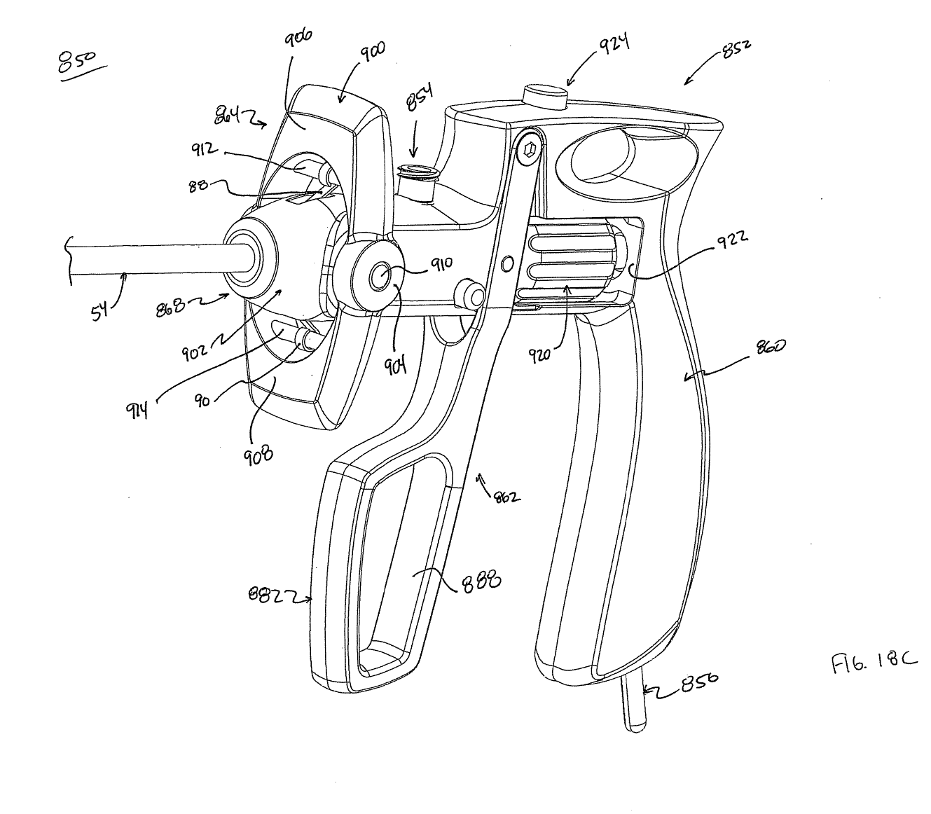

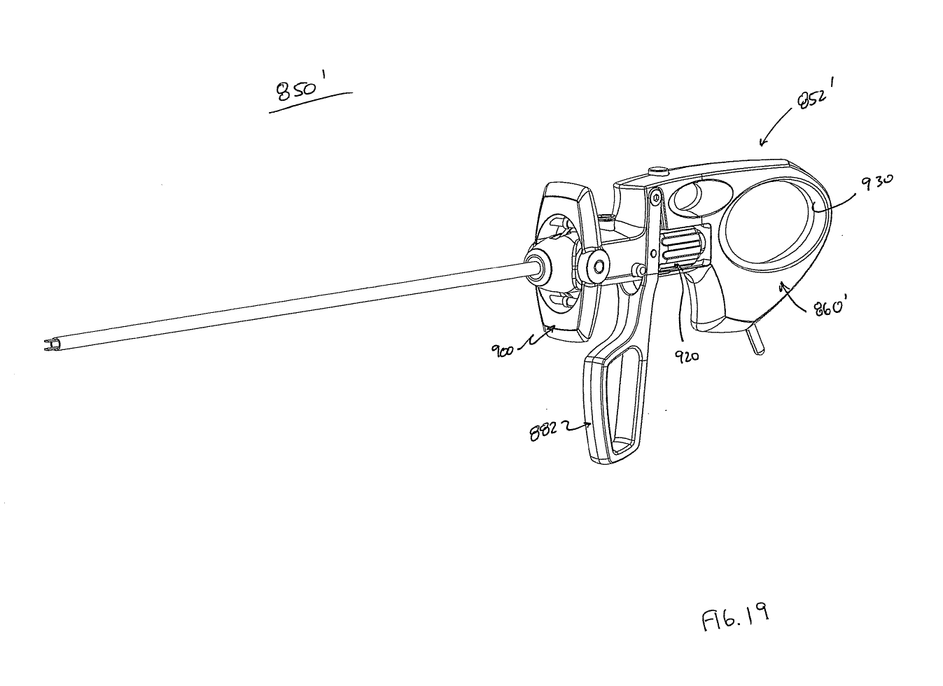

Another embodiment articulating laparoscopic surgical instrument 850 in accordance with principles of the present disclosure is shown in FIGS. 18A-18C. As with previous embodiments, the instrument 850 includes the end effector 52, the shaft 54, a handle assembly 852, an optional flush port assembly 854, and an optional cautery post 856. As a point of reference, the end effector 52 is omitted from the view of FIG. 18A, as is the deflection assembly 58 formed or carried by the shaft 54. Thus, in the view of FIG. 18A, the cables 88, 90 are illustrated as extending from the shaft 54. Conversely, in the view of FIG. 18B, the flush port assembly 854, the cautery post 856, and a portion of the handle assembly 852 (and in particular a handle body 860) are omitted to better illustrate various internal components. Further, a portion of the shaft 54 adjacent the handle assembly 852 is cut away to better illustrate the cables 88, 90. Finally, FIG. 18C provides an enlarged view of the handle assembly 852 of FIG. 18A.

In addition to the handle body 860, the handle assembly 852 includes components of the various mechanisms described above, including an end effector operation mechanism 862, an articulation mechanism 864, an end effector rotation mechanism 866, and a shaft rotation mechanism 868. The mechanisms 862-868 are referenced generally in the view of FIG. 18B and effectuate instrument operations in accordance with previous embodiments, facilitating user-controlled operation and rotation of the end effector 52, articulation of the deflection assembly 58, and optionally rotation of the shaft 54.

With reference to FIGS. 18B and 18C, the end effector operation mechanism 862 includes a rod 880, an end effector operation actuator 882, and a slide body 884. The rod 880 is similar to previous embodiments (e.g., the rod 130 (FIG. 5) described above), and defines a proximal portion coupled to the slide body 884 as described below. Further, the rod 880 extends through the shaft 54 and is coupled to the end effector 52 such that an operational arrangement of the end effector 52 is altered with longitudinal movement (i.e., push/pull) of the rod 880.

The end effector operation actuator 882 can assume various forms, and with the one embodiment of FIG. 18C is akin to a trigger that combines with the handle body 860 to form a pistol grip-like structure. In more general terms, the actuator 882 is pivotably coupled to the handle body 860, and forms one or more features (e.g., a finger ring 888) adapted to facilitate user handling and interface.

With specific reference to FIG. 18B, the slide body 884 establishes a link between the rod 880 and the trigger actuator 882 whereby movement of the trigger actuator 882 relative to the handle body 860 applies a pushing or pulling force (in the longitudinal direction) onto the rod 880. For example, the slide body 884 can include a first leg 890 attached to and extending from the actuator 882, and a second leg 892 projecting from the first leg 890 and coupled to the rod 880. The rod 880/slide body 884 coupling can assume any of the forms previous described, for example the second leg 892 forming a slot 894 within which the rod 880 is rotatably maintained. Hubs 896 (one of which is referenced generally in FIG. 18B) are formed or provided along the rod 880 at opposite sides of the second leg 892. As with previous embodiments, this construction translates a user-applied force on the actuator 882 into a longitudinal force applied to the rod 880, yet permits rotation of the rod 880 relative to the slide body 884. Other coupling configurations are also envisioned.

With reference to FIGS. 18B and 18C, the articulation mechanism 864 includes a paddle 900 pivotably retained relative to an exterior of the handle body 860. The paddle 900 serves as an actuator of the articulation mechanism 864, and acts upon the cables 88, 90 and thus the deflection assembly 58 (that can also be considered components of the articulation mechanism 864). The paddle 900 is akin to the pivot or articulation arm 530 (FIG. 13C) described above, except that the paddle 900 is external the handle body 860. For reasons made clear below, the paddle 900 is pivotably attached to a knob 902 that in turn is rotatably coupled with the handle body 860. In this regard, the paddle 900 includes or defines a central section 904 and opposing end sections 906, 908. Mounting of the central section 904 to the knob 902 (or other component associated with the handle body 860) establishes a pivot point 910. The end sections 906, 908 project from opposite sides of the pivot point 910, and are secured to a respective one of the cables 88, 90. For example, the first cable 88 is attached to the first end section 906 (e.g., by a pin 912), and the second cable 90 is attached to the second end section 908 (e.g., by a pin 914). With this construction, a user-caused pivoting movement of the paddle 900 alters, in an opposing fashion, tension in the cables 88, 90. For example, relative to the orientation of FIGS. 18A-18C, rotation of the paddle body 900 in a clockwise direction increases a tension or force in the first cable 88 and lessens, by an equal amount, tension in the second cable 90. Commensurate with the above descriptions, this change in cable tension effectuates a more or less dramatic curve at the deflection assembly 58. The articulation mechanism 864 is mechanically isolated from the rod 880 such that movement or toggling of the paddle 900 does not directly transmit a force onto the rod 880, nor does the articulation mechanism 864 interfere with desired operation of the effector operation mechanism 862.

Optionally, the handle assembly 852 can incorporate cleaning mode features in which a coupling between the paddle 900 and the knob 902 is selectively releaseable (e.g., a switch or similar device is provided that permits a user to readily disconnect and re-assemble the paddle 900 to the knob 902). When released (i.e., cleaning mode), the paddle body 900 can be moved longitudinally forward relative to the knob 902 to create slack in the cables 88, 90. This slack, in turn, can facilitate separation of components of the deflection assembly 58 for cleaning purposes as described above.

The end effector rotation mechanism 866 includes an end effector rotation actuator 920 coupled to the rod 880. The actuator 920 can be akin to a thumb wheel that is, in some embodiments, rotatably maintained by the handle body 860. While the actuator 920 is shown as being directly attached to the rod 880, in other constructions one or more intermediate gears can be interposed between the rod 880 and the actuator 920. Regardless, user-caused rotation of the thumb wheel actuator 920 results in rotation of the end effector 52 relative to the shaft 54 via rotation of the rod 880. Notably, an arrangement of the thumb wheel actuator 920 relative to the handle body 860 and the rod 880 permits longitudinal movement of the rod 880 (i.e., during operation of the end effector operation mechanism 862) while maintaining the rotational force-transferring relationship between the rod 880 and the actuator 920. For example, the thumb wheel actuator 920 can be affixed to the rod 880 and disposed within an open channel 922 formed by the handle body 860; the thumb wheel actuator 920 moves longitudinally within the channel 922 with sliding movement of the rod 880. Alternatively, a sliding gear-type arrangement can be established between the rod 880 and the thumb wheel actuator 920, akin to the relationship described above with respect to the gears 584, 586 (FIG. 13B). The end effector rotation mechanism 866 optionally further includes a locking device 924 (referenced generally in FIG. 18C) configured to allow a user to selectively lock the thumb wheel actuator 920 (and thus the rod 880 and the end effector 52) at a desired rotational arrangement.

The shaft rotation mechanism 868 includes the knob 902 mentioned above. The knob 902 is fixed to the shaft 54, and serves as a shaft rotation actuator. As previously described, the knob 902 is rotatably coupled to the handle body 860, such that rotation of the knob 902 causes the shaft 54 to rotate relative to the handle body 860. As with previous embodiments, the shaft rotation mechanism 868 is mechanically isolated from the rod 880, such that rotation of the shaft 54 does not directly cause or result in rotation of the rod 880. Further, with the embodiment of FIGS. 18A-18C, the paddle 900 is pivotably coupled to the knob 902, thereby establishing a rotatably fixed connection between the knob 902 and the cables 88, 90 via the paddle 900. Thus, a rotational force applied to the knob 902 and/or the paddle 900 causes the cables 88, 90 to rotate with rotation of the shaft 54, limiting possible binding or twisting of the cables 88, 90.

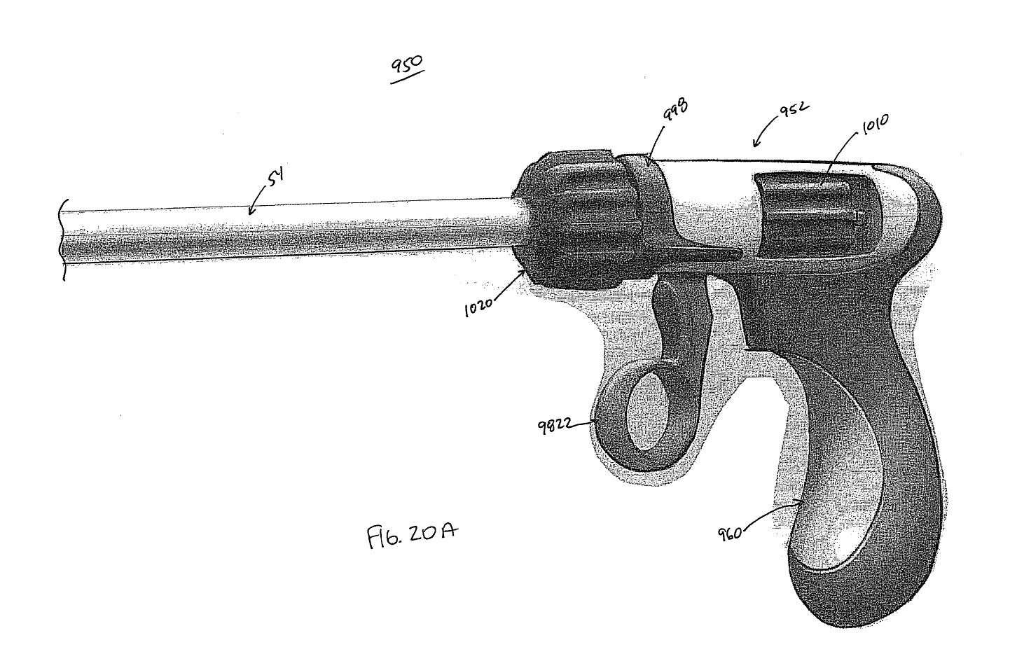

The handle assembly 852 can assume a variety of other shapes and constructions conducive to convenient user handling. In more general terms, by locating the paddle body/articulation mechanism actuator 900 at an exterior of the handle body 860, the instrument 850 provides a user with direct visual understanding of a curvature or bend in the deflection assembly 58 under circumstances where the deflection assembly 58 is otherwise located within the patient and thus not directly viewable. A related embodiment instrument 850′ is shown in FIG. 19, and is virtually identical to the instrument 850 (FIG. 18A) described above, except that a handle body 860′ provided with handle assembly 852′ has a differing shape. With the handle body 860′ of FIG. 19, a hole 930 is formed through the handle body 860′, and provides a convenient location for a user's thumb. With this handling technique, then, the user's fingers may more readily interface with the end effector operation actuator/trigger 882, the paddle 900, and the end effector rotation actuator/thumb wheel 920.

Handle Assembly with Lead Screw

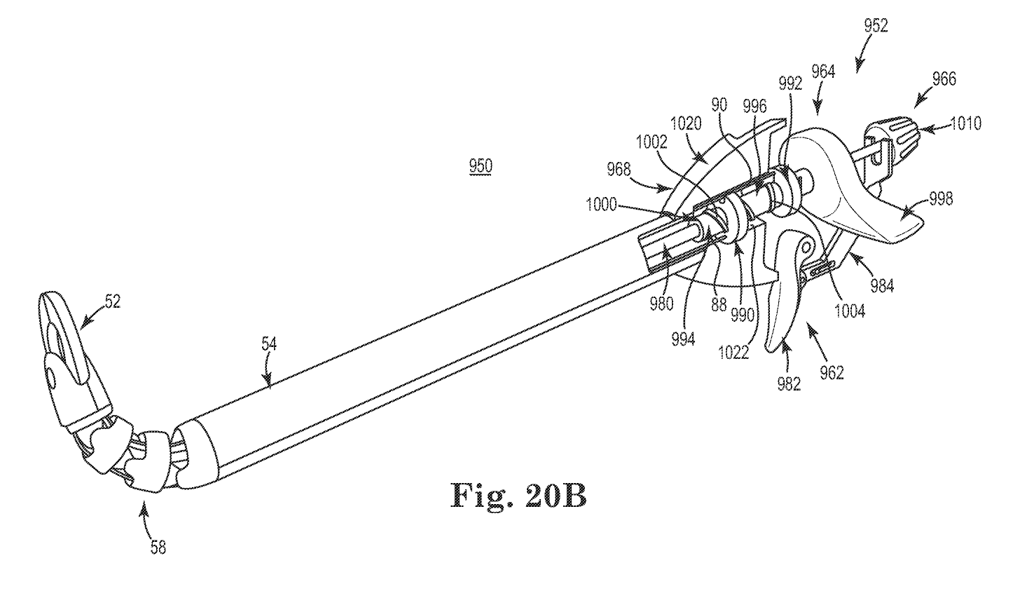

Another embodiment articulating laparoscopic surgical instrument 950 in accordance with principles of the present disclosure is shown in FIGS. 20A and 20B. As with previous embodiments, the instrument 950 includes the end effector 52, the shaft 54, and a handle assembly 952. As a point of reference, the end effector 52 and a portion of the shaft 54 are omitted from the view of FIG. 20A for ease of illustration. Conversely, a handle body 960 portion of the handle assembly 952 is omitted from the view of FIG. 20B to better illustrate various components of an end effector operation mechanism 962, an articulation mechanism 964, an end effector rotation mechanism 966, and a shaft rotation mechanism 968 provided with the handle assembly 952.

The end effector operation mechanism 962 includes a rod 980, an end effector operation actuator 982, and a slide body 984. The rod 980 can assume any of the constructions previously described (i.e., the rod 130 of FIG. 5), and forms a proximal portion 986 that is rotatably coupled with the slide body 984. The actuator 982 is linked to the rod 980 via the slide body 984 in accordance with previous descriptions. Further, the end effector operation actuator 982 is pivotably coupled with the handle body 960. In this regard, while the actuator 982 is illustrated as being akin to a trigger (with the trigger actuator 982 and the handle body 960 combining to define a pistol grip-like shape) other constructions are also envisioned. In more general terms, then, a user applied force on the actuator 982 results in a longitudinal movement of the rod 980, and thus a change in the operational arrangement of the end effector 52. However, the rod 980/slide body 984 coupling permits rotational movement of the rod 980 relative to the slide body 984.

The articulation mechanism 964 includes first and second discs or collars 990, 992, first and second lead screw bodies 994, 996, and an articulation actuator 998. The first cable 88 is attached to the first disc 990, and the second cable 90 is attached to the second disc 992. Thus, in some embodiments, the cables 88, 90 and the deflection assembly 58 can be considered “part” of the articulation mechanism 964.

The lead screw bodies 994, 996 are formed along a common tube 1000 that is coaxially disposed over the rod 980. The lead screws 994, 996 and the tube 1000 are comprised of a structurally robust material, such as steel. Each of the lead screw bodies 994, 996 forms a thread 1002, 1004. The threads 1002, 1004 have an identical (or nearly identical) angle and pitch, but are oriented in opposite directions. For example, the thread 1002 of the first lead screw body 994 is a left-handed thread, whereas the thread 1004 of the second lead screw body 996 is a right-handed thread (or vice-versa). The first disc 990 is mounted over the first lead screw body 994 and threadably engages the thread 1002. The second disc 992 is mounted over the second lead screw body 996 and threadably engages the thread 1004. Finally, the articulation actuator 982 has a paddle-like shape as shown, and is fixed to the tube 1000. With this construction, rotation of the paddle actuator 982 in a first direction causes the discs 990, 992 to move longitudinally away from one another as they interface with the corresponding, rotating thread 1002, 1004. Conversely, rotation of the paddle 982 in the opposite direction causes the discs 980, 982 to move longitudinally toward one another. Because the threads 1002, 1004 have an identical or nearly identical angle and pitch, the rate of movement or distance traveled with rotation of the paddle actuator 982/tube 1000 is substantially identical for the discs 990, 992. Thus, and as with pervious embodiments, longitudinal movement of the discs 990, 992 alters tension in the corresponding cables 88, 90 in an equal but opposite fashion. This change in tension, in turn, alters the curvature formed in the deflection assembly 58. Notably, the lead screw bodies 994, 996 and the tube 1000 are mechanically isolated from the rod 980. Thus, operation of the articulation mechanism 964 does not directly act upon the rod 980; further, the articulation mechanism 964 does not impede operation of the end effector operation mechanism 962 (or the end effector rotation mechanism 966 described below).

The end effector rotation mechanism 966 includes an actuator 1010 and the rod 980, with the end effector rotation actuator 1010 being coupled to the proximal portion 986 of the rod 980. The actuator 1010 can be akin to a thumb wheel, and is directly or indirectly linked to the rod 980. Further, the thumb wheel actuator 1010 is arranged relative to the handle body 960 so as to permit user-caused rotation thereof, as well as to permit longitudinal movement of the rod 980 with operation of the articulation mechanism 964. For example, though hidden in the view of FIG. 20A, an internal gear formed by the thumb wheel actuator 1010 meshes with an exterior gear formed or provided along the rod 980. The so-provided geared interface allows the rod 980 to longitudinally slide relative to the thumb wheel actuator 1010 while maintaining rotational engagement therebetween. Other constructions are also envisioned. For example, as implicated by the schematic illustration of FIG. 20B, the thumb wheel actuator 1010 can be directly linked to the rod 980, with the handle body 960 being constructed to permit the thumb wheel actuator 1010 to slide with longitudinal movement of the rod 980.

The shaft rotation mechanism 968 is akin to previous descriptions, and includes a shaft rotation actuator 1020 in the form of a knob. The knob 1020 is fixed to the shaft 54, and is rotatably mounted to the handle body 960. With this construction, user-imparted rotation of the knob 1020 is transferred to the shaft 54 to effectuate rotation of the shaft 54 relative to the handle body 960. The knob 1020 forms a cavity 1022 within which the discs 990, 992 and the lead screw bodies 994, 996 are disposed. Further, the cables 88, 90, and the rod 980 extend through and distally beyond the cavity 1022. The cavity 1022 is sized and shaped to permit longitudinal movement of the discs 990, 992 along the tube 1000 as described above during operation of the articulation mechanism 964. With the one construction of FIG. 20B, then, the discs 990, 992 are not directly linked to the knob 1020. Rotation of the knob 1020 is thus not transferred onto the discs 990, 992. As a result, during rotation of the shaft 54 (via manipulation of the knob 1020), the cables 88, 90 are not directly caused to rotate, and may slightly twist. In other embodiments, however, a link can be established between the cables 88, 90 and the knob 1020 (e.g., such as described above with respect to the articulation mechanism 464 of FIG. 10B).

The articulating laparoscopic surgical instruments of the present disclosure provide marked improvements over previous designs. All operational features desired by surgeons for performing a single incision laparoscopic procedure are facilitated by one or more actuators situated along the instrument's handle. In many embodiments, a single hand of the user can grip the instrument handle and operate the end effector, articulate or bend the shaft (or a deflection assembly associated with the shaft), rotate the end effector, and rotate the shaft. Further, instruments of the present disclosure are uniquely designed for reuse, including cleaning and sterilization. In this regard, an optional flush port assembly is included to facilitate cleaning and sterilization of the instrument shaft.

Although the present disclosure has been described with reference to preferred embodiments, workers skilled in the art will recognize that changes can be made in form and detail without departing from the spirit and scope of the present disclosure.

What is claimed is:

1. An articulating surgical instrument comprising:
a handle body;
an elongated shaft extending from the handle body to a shaft end;
an end effector connected to the shaft end, the end effector including a first body movably associated with a second body;
an end effector operation mechanism including:
a rod defining a proximal portion maintained by the handle body and a distal portion extending from the handle body and through the shaft, a distal end of the rod coupled to the end effector such that longitudinal movement of the rod causes the first body to move relative to the second body,
an end effector operation actuator movably coupled to the handle body and linked to the proximal portion of the rod,
wherein movement of the actuator relative to the handle body transfers a force onto the rod in a longitudinal direction; and
an articulation mechanism including:
a deflection assembly disposed over at least a segment of the rod distal portion and configured to bend and straighten the segment,
first and second collar assemblies slidably disposed over the rod proximal portion, the first collar assembly being longitudinally spaced from the second collar assembly,
a first cable extending between the first collar assembly and the deflection assembly,
a second cable extending between the second collar assembly and the deflection assembly,
a linkage interconnecting the first and second collars,
an articulation actuator coupled to the linkage and movably connected to the handle body,
wherein the articulation mechanism is configured such that movement of the articulation actuator relative to the handle body moves the first and second collars in opposite directions to cause a longitudinal deflection of the deflection assembly.

2. The surgical instrument of claim 1, wherein the first and second cables extend along opposing sides of the shaft.

3. The surgical instrument of claim 1, wherein the linkage is configured to simultaneously move the first and second collar assemblies an equal distance in response to movement of the articulation actuator.

4. The surgical instrument of claim 1, wherein the linkage is configured such that movement of the articulation actuator in a first direction causes the collar assemblies to move toward one another, and movement of the articulation actuator in a second, opposite direction causes the collar assemblies to move away from one another.

5. The surgical instrument of claim 1, wherein the first collar assembly is arranged distal the second collar assembly, and further wherein the first collar assembly forms a passageway through which the second cable extends.

6. The surgical instrument of claim 1, wherein the linkage includes:
a first drive arm connected to the first collar assembly;
a second drive arm connected to the second collar assembly; and
a pivot arm connected to each of the first and second drive arms opposite the corresponding collar assembly;
wherein the pivot arm is further connected to the articulation actuator and is rotatably maintained by the handle body.

7. The surgical instrument of claim 6, wherein movement of the articulation actuator causes the pivot arm to rotate, and further wherein rotation of the pivot arm simultaneously applies a first direction force on the first drive arm and a second, opposite direction force on the second drive arm.

8. The surgical instrument of claim 7, wherein the pivot arm rotates about a center point intersecting the rod.

9. The surgical instrument of claim 6, wherein the pivot arm forms opposing, first and second slots and is arranged such that the slots are located at opposite sides of the rod.

10. The surgical instrument of claim 9, wherein the first drive arm is attached to a first pin slidably captured within the first slot, and the second drive arm is attached to a second pin slidably captured within the captured within the second slot.

11. The surgical instrument of claim 10, wherein the first pin slides within the first slot and the second pin slides within the second slot with rotation of the pivot arm.

12. The surgical instrument of claim 10, wherein the articulation actuator includes a thumb switch slidably mounted to the handle body and a post attached to the thumb switch and slidably captured within the first slot.

13. The surgical instrument of claim 10, wherein the articulation actuator includes a thumb wheel rotatably mounted to the handle body, the thumb wheel including gear teeth meshingly engaging a gear extending from the pivot arm.

14. The surgical instrument of claim 1, wherein each of the collar assembly includes:
a first collar member attached to the corresponding cable; and
a second collar member attached to the linkage;
wherein the first collar member is rotatably mounted over the second collar member.

15. The surgical instrument of claim 14, wherein with respect to each of the collar assemblies:
a longitudinal force applied to the second collar member is transferred to the first collar member; and
the first collar member freely rotates relative to the second collar member.

16. The surgical instrument of claim 15, further comprising:
a shaft rotation actuator knob coupled to the shaft and forming a cavity maintaining the first and second collar assemblies, including the first collar members being longitudinally slidable along the cavity and rotationally fixed to the shaft rotation actuator knob;
wherein the shaft rotation actuator knob is rotatably coupled to the handle body such that shaft rotation of the rotation actuator knob cause a corresponding rotation of the shaft, the first collar members, and the cables relative to the handle body.

17. The surgical instrument of claim 16, wherein the first collar member rotates relative to the second collar member with rotation of the shaft rotation actuator knob.

18. The surgical instrument of claim 1, further comprising an end effector rotation mechanism including:
an end effector rotation actuator rotatably maintained by the handle body and connected with the proximal portion of the rod;
wherein the end effector rotation mechanism is configured such that rotation of the end effector rotation actuator causes the end effector to spatially rotate via caused rotation of the rod.

19. The surgical instrument of claim 18, wherein the rod rotates relative to the collar assemblies with rotation of the end effector rotation actuator.

20. The surgical instrument of claim 19, wherein the proximal portion forms a first gear and the end effector rotation mechanism includes a second gear extending from the end effector rotation actuator, and further wherein the first gear meshingly engages the second gear in a manner permitting longitudinal sliding movement of the rod relative to the end effector rotation actuator in response to actuation of the end effector operation actuator.

21. The surgical instrument of claim 1, wherein the linkage includes a pivot arm defining first and second end sections at opposite sides of a central section, a first drive arm connecting the first collar assembly and the first end section, and a second drive arm connecting the second collar assembly and the second end section, and further wherein the instrument is configured to provide:
a use mode in which the central section is pivotably mounted relative to the shaft at a pivot point in a manner preventing longitudinal movement of the pivot point relative to the shaft; and
a cleaning mode in which the pivot point is longitudinally de-coupled relative to the shaft, permitting forward longitudinal movement of both of the collar assemblies to generate slack in the cables.

22. An articulating surgical instrument comprising:
a handle body;
an elongated shaft extending from the handle body to a shaft end;
an end effector connected to the shaft end, the end effector including a first body movably associated with a second body;
an end effector operation mechanism including:
a rod defining a proximal portion maintained by the handle body and a distal portion extending from the handle body and through the shaft, a distal end of the rod coupled to the end effector such that longitudinal movement of the rod causes the first body to move relative to the second body,
an end effector operation actuator movably coupled to the handle body and linked to the proximal portion of the rod,
wherein movement of the actuator relative to the handle body transfers a force onto the rod in a longitudinal direction;
a deflection assembly disposed over at least a segment of the rod distal portion and configured to bend and straighten the segment;
a knob rotatably coupled to the handle body and rotationally fixed to the shaft;
a pivot arm disposed within a cavity formed by the knob, the pivot arm being pivotably coupled to the shaft at a pivot point such that the pivot arm defines opposing, first and second end sections at opposite sides of the pivot point;
a first cable extending between the first end section and the deflection assembly; and
a second cable extending between the second end section and the deflection assembly;
wherein the instrument is configured such that longitudinal movement of the knob relative to the shaft causes the pivot arm to pivot about the pivot point and apply opposing forces onto the cables to cause a longitudinal deflection of the deflection assembly;
and further wherein rotation of the knob is transferred to the shaft to cause rotational movement of the shaft relative to the handle body.

23. An articulating surgical instrument comprising:
a handle body;
an elongated shaft extending from the handle body to a shaft end;
an end effector connected to the shaft end, the end effector including a first body movably associated with a second body;
an end effector operation mechanism including:
a rod defining a proximal portion maintained by the handle body and a distal portion extending from the handle body and through the shaft, a distal end of the rod coupled to the end effector such that longitudinal movement of the rod causes the first body to move relative to the second body,
an end effector operation actuator movably coupled to the handle body and linked to the proximal portion of the rod,
wherein movement of the actuator relative to the handle body transfers a force onto the rod in a longitudinal direction; and
an articulation mechanism including:
a deflection assembly disposed over at least a segment of the rod distal portion and configured to bend and straighten the segment,
a paddle pivotably coupled to an exterior of the handle body at a pivot point, the paddle defining opposing first and second end sections at opposite sides of the pivot point,
a first cable extending between the first end section and the deflection assembly along a first side of the shaft,
a second cable extending between the second end section and the deflection assembly along an opposite, second side of the shaft,
wherein the articulation mechanism is configured such that pivoting of the paddle relative to the handle body applies opposing forces to the cables to cause a longitudinal deflection of the deflection assembly.
